(19) United States
(12) Patent Application Publication
(10) Pub. No.: US 2006/0160784 A1
(43) Pub. Date: Jul. 20, 2006

(54) Method for treating neurologic diseases

(75) Inventors: Darren Magda, Cupertino, CA (US); Richard Miller, Portola Valley, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc.

(21) Appl. No.: 11/337,187

(22) Filed: Jan. 19, 2006

Related U.S. Application Data
(60) Provisional application No. 60/645,681, filed on Jan. 19, 2005.

(51) Int. Cl. A61K 31/555

(52) U.S. Cl. 514/185

(57) ABSTRACT

Disclosed herein are texaphyrin-metal complexes, compositions comprising such complexes, pharmaceutical formulations comprising such complexes, and methods for treating neurologic diseases, disorders and conditions and or free-radical associated diseases, disorders and conditions using such complexes, compositions and pharmaceutical formulations.

### CROSS-REFERENCE TO RELATED APPLICATIONS

This Application claims the benefit of U.S. Provisional Application No. 60/645,681, filed on Jan. 19, 2005, the disclosure of which is hereby incorporated by reference in its entirety.

### FIELD OF THE INVENTION

Disclosed herein are texaphyrin metal complexes and the use of such texaphyrin metal complexes, alone or in combination, to treat neurologic diseases, disorders and conditions.

### BACKGROUND OF THE INVENTION

Amyotrophic lateral sclerosis (ALS or Lou Gerhig's Disease) is a fatal neurodegenerative disease which typically strikes in the prime of life. The average age of onset follows a bell-shaped probability curve with a peak at approximately 45-50 years old and the time from the beginning of symptoms to death ranges from 1-6 years. Other than possible genetic predictors for familial inheritance of the disease, there are no general predictors of sporadic ALS and no way to know who is at risk prior to the onset of symptoms. Moreover, differential diagnosis often doesn't occur until weeks or months after the first symptoms. Thus, any potential treatments for ALS have been aimed at slowing disease progression and preserving the remaining spinal motor neurons.

### SUMMARY OF THE INVENTION

In one aspect are methods for treating neurological (neurologic) diseases, disorders and conditions and/or free-radical associated diseases, disorders and conditions comprising administration of a texaphyrin metal complex having the structure of Formula (I): 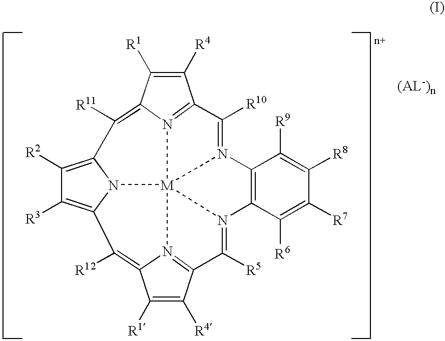
wherein:
M is a transition metal ion or a lanthanide metal ion,
AL is an apical ligand;
n is 1, 2, 3, 4, or 5;
R⁶ and R⁹ are independently chosen from the group: acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, sulfanyl, sulfinyl, sulfonyl, and the moiety —X-Y where: X is a covalent bond or a linker, and Y is a catalytic group, a neuroprotectiv agent or a site-directing group;
R¹, R^{1′}, R², R³, R⁴, R⁴′, R⁷ and R⁸ are independently chosen from the group: acyl, acyloxy, alkyl, optionally substituted alkenyl, optionally substituted alkoxy, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, sulfanyl, sulfinyl, sulfonyl, and the moiety —X-Y where: X is a covalent bond or a linker, and Y is a catalytic group, a neuroprotective agent or a site-directing group; and
R⁵, R¹⁰, R¹¹ and R¹² are independently chosen from the group: acyl, optionally substituted alkoxy, optionally substituted alkyl, optionally substituted aryl, halo, and hydrogen;
   with the proviso that for R⁶ and R⁹, halogen is other than iodide and substituted alkyl is other than iodoalkyl; and with the proviso that at least one of R¹, R^{1′}, R², R³, R⁴, R⁴′, R⁷ and R⁸ is —O-(optionally substituted alkylene-O)ₙ-alkyl, where n is 1, 2, 3, 4, 5, 6, 7, 8, 9, or 10.

In further or alternative embodiments, at least two of R¹, R^{1′}, R², R³, R⁴, R^{4′}, R⁷ and R⁸ are —O-(optionally substituted alkylene-O)ₙ-alkyl, where n is 1, 2, 3, 4, 5, 6, 7, 8, 9, or 10. In further or alternative embodiments, R⁵, R¹⁰, R¹¹ and R¹² are H. In further or alternative embodiments, at least two of R¹, R^{1′}, R², R³, R⁴, R⁴′, R⁷ and R⁸ are unsubstituted alkyl. In further or alternative embodiments, at least four of R¹, R^{1′}, R², R³, R⁴, R⁴′, R⁷ and R⁸ are unsubstituted alkyl. In further or alternative embodiments, AL is derived from any molecule containing a carboxylic acid or phosphate group. In further or alternative embodiments, AL is acetate. In further or alternative embodiments, M is selected from the group consisting of lanthanum, cerium, praseodymium, neodymium, promethium, samarium, europium, gadolinium, terbium, dysprosium, holmium, erbium, thulium, ytterbium and lutetium. In further or alternative embodiments, M is Ce(III), Sm(II), Sm(III), Eu(II), Eu(III), Gd(III), Yb(II), Yb(III) and Lu(III). In further or alternative embodiments, M is selected from titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, ununnilium, unununium, or ununbium.

In further or alternative embodiments, R⁷ and R⁸ are —O-(alkylene-O)ₙ-alkyl, where n is 1, 2, 3, 4, 5, 6, 7, 8, 9, or 10. In still further or alternative embodiments, n is an integer selected from 2, 3, 4, or 5. In even further or alternative embodiments, n is 3. In further or alternative embodiments, R⁶ and R⁹ are hydrogen. In further or alternative embodiments, R⁵, R¹⁰, R¹¹ and R¹² are hydrogen.

In further or alternative embodiments, the texaphyrin-metal complex has the structure: 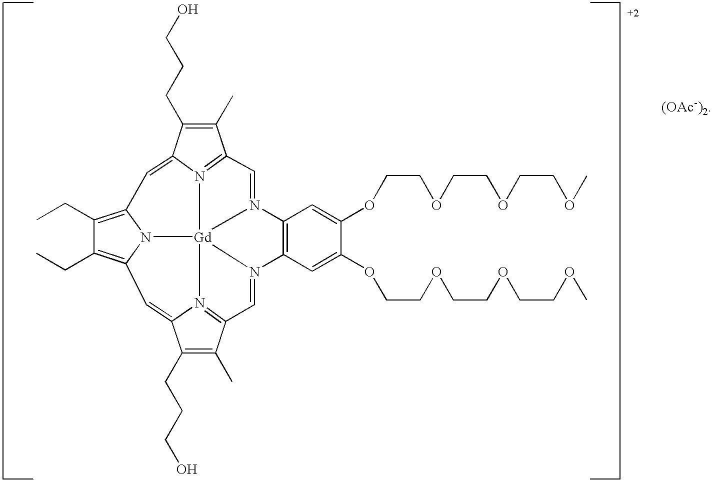
In further or alternative embodiments, the texaphyrin-metal complex has the structure:

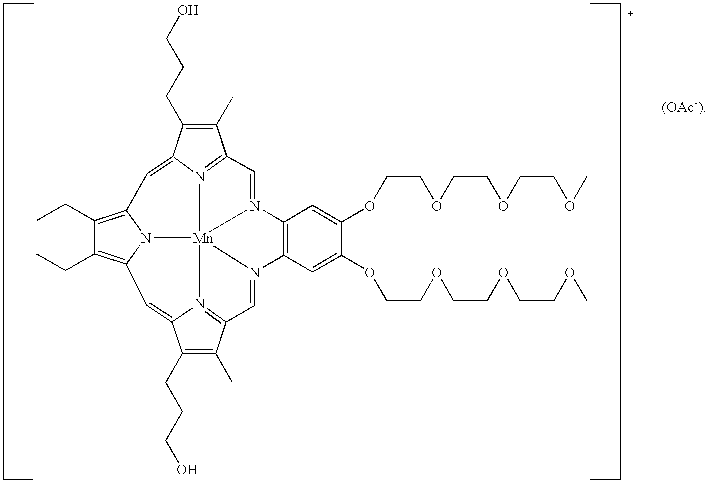

In further or alternative embodiments, the texaphyrin-metal complex decreases intracellular reactive oxygen species. In further or alternative embodiments, the reactive oxygen species is OH, H₂O₂, O₂.⁻, NO., or ⁻OONO. In further or alternative embodiments, the presence of such reactive oxygen species is associated with a disease. In further or alternative embodiments, the administration of such texaphyrin-metal complexes results in the prevention, arresting or treatment of such diseases associated with such reactive oxygen species. In further or alternative embodiments, such diseases are dementia, Lou Gerhig's disease, motor neurone disorders, dermatitis, delayed type hypersensitivity, multiple organ failure, allergic rhinitis, pneumonia, emphysema, chronic bronchitis, AIDS, pancreatitis, hypertension, congestive heart failure, angioplasty, endocarditis, retinopathy of premanurity or uveitis. In further or alternative embodiments, such diseases are amyotrophic lateral sclerosis (ALS), Alzheimer's disease, Parkinson's disease, multiple sclerosis (MS), Huntington's disease, arthritis, or radiation toxicity. In further or alternative embodiments, the texaphyrin-metal complex has myocardial protective activity, skeletal muscle protective activity, or cerebral protective activity. In further or alternative embodiments, the texaphyrin-metal complex is administered in a solution. In further or alternative embodiments, the texaphyrin-metal complex is administered intravenously. In further or alternative embodiments, the texaphyrin-metal complex is administered in a solution containing about 2-8% of mannitol. In further or alternative embodiments, the pH of the texaphyrin-metal complex solution is between about 5 and 6.

In further or alternative embodiments, the texaphyrin-metal complex is administered in multiple doses. In further or alternative embodiments, the texaphyrin-metal complex is co-administered with an antiemetic. In further or alternative embodiments, the patient is further co-administered with an agent selected from a thrombolytic agent, an anti-anginal agent a reducing agent, another neurological therapeutic agent, or a zinc compound. In further or alternative embodiments, the patient is further administered with an agent selected from a thrombolytic agent, an anti-anginal agent a reducing agent, another neurological therapeutic agent, or a zinc compound.

In one embodiment the compound of Formula (I) has at least one of the following properties: (a) M is a transition metal; (b) M is a lanthanide metal; (c) the compound of Formula (I) comprises at least one polyethylene glycol moiety; (d) the compound of Formula (I) comprises at least one poly-hydroxylated group; (e) the compound of Formula (I) is metallated with Gd(III); (f) the compound of Formula (I) is metallated with Lu(III); (g) the compound of Formula (I) is synthesized from a tripyrrane moiety; (h) the compound of Formula (I) is asymmetrically substituted; (i) the substitution pattern of the compound of Formula (I) has a mirror symmetry; (j) the compound of Formula (I) includes a further neuroprotective agent; (k) the compound of Formula (I) is substituted with at least 1 methyl group; or (1) at least 95%, at least 96%, at least 97%, at least 98%, or at least 99% of the compound of Formula (I) is in the composition have the same molecular weight. In further aspects are compositions in which the compound of Formula (I) has at least two of the aforementioned properties; in further aspects, at least three of the aforementioned properties; in further aspects, at least four of the aforementioned properties; and in further aspects, at least five of the aforementioned properties.

In another embodiment are formulations for treating a neurologic disease, disorder or condition, and/or a free-radical associated disease, disorder or condition comprising a compound of Formula (I), wherein the formulation has at least one of the following characteristics (a) the compound of Formula (I) is selected from one of the aforementioned compounds having a structure of Formula (I); (b) the formulation is suitable for administration to a mammal; (c) the formulation is suitable for administration to a human; (d) the formulation is suitable for administration to a human patient having a neurodegenerative disease or disorder; (e) the formulation is suitable for administration to a patient having a neurodegenerative disease or disorder; (f) the formulation is suitable for administration to a patient having ALS; (g) the formulation is suitable for administration to a patient having dementia; (h) the formulation is suitable for administration to a patient having a motor neurone disorder; (i) the formulation is suitable for administration to a patient having multiple organ failure; (j) the formulation is suitable for administration to a patient having ischemia; (k) the formulation is suitable for administration to a patient having AIDS; (l) the formulation is suitable for administration to a patient having multiple sclerosis; (m) the formulation is suitable for administration to a patient having Parkinson's disease; (n) the formulation contains pharmaceutically acceptable excipients; (o) the formulation is in the form of a pharmaceutically-acceptable solid dosage form; (p) the formulation is in the form of a pharmaceutically-acceptable non-solid dosage form; (q) the formulation is in the form of a pharmaceutically-acceptable suspension; (r) the formulation further comprises water; (s) the formulation further comprises acetic acid; (t) the formulation is in the form of an intravenously-suitable formulation; (u) the formulation is in the form of a pharmaceutically-acceptable solution; (v) the formulation is in the form of a pharmaceutically-acceptable suppository; (w) the formulation is in the form of a pharmaceutically-acceptable tablet or capsule; (x) the formulation does not comprise a preservative; (y) the formulation is suitable for administration to a patient via a route selected from oral, rectal intranasal, intra-arterial, intraperitoneally, parenterally, topical, subcutaneous, intramuscular, buccal, intravenous, trandermal, inhaled, or via an impregnated or coated device; (z) the formulation is in the form of a prodrug; (aa) the formulation contains a pharmaceutically acceptable salt, (ab) the formulation may be administered in either single or multiple doses; (ac) the formulation comprises mannitol; (ad) the formulation contains at least one anti-aggregation agent; (ae) the formulation does not comprise an oxidizing agent other than the compound of Formula (I); (af) the formulation is formulated in a unit dosage form; (ag) the formulation may be administered for photodynamic therapy; (ah) the formulation may be administered with other neuroprotective drugs; (ai) the formulation may be administered before, at the same time as, or after administration of one or more neuroprotective drugs; (aj) the formulation is administered for radiation sensitization; (ak) the formulation is administered for sonodynamic therapy; (al) the formulation is administered before administration of ultrasound; (am) the formulation is administered in a combination therapy; (an) the formulation is administered to a patient in conjunction with at least one anti-inflammatory agent; (ao) the formulation is administered to a patient in conjunction with at least one zinc reagent, (ap) the formulation is administered in conjunction with another neurological therapeutic agents, (aq) the formulation is packaged in a container that is packaged in a cardboard box; (ar) the formulation is packaged in a bottle; (as) the formulation is packaged in container wherein the headspace comprises less than about 10% oxygen; (at) the stored formulation is stable for at least three years, (au) the formulation is neuroprotective, and (av) the formulation is cerebral protective, wherein stability means that the formulation contains less than about 30 ppm of gadolinium ions that are not complexed by an compound of Formula (I). In further aspects are formulations in which the formulation has at least two of the aforementioned characteristics; in further aspects, at least three of the aforementioned characteristics; in further aspects, at least four of the aforementioned characteristics; and in further aspects, at least five of the aforementioned characteristics.

In another aspect are methods for treating a neurologic disease, disorder or condition, and/or a free-radical associated disease, disorder or condition, in a patient comprising administering a formulation comprising an compound of Formula (I), wherein the method includes at least one of the following steps or characteristics: (a) the patient is administered at least one of the aforementioned compound of Formula (I) formulations; (b) the disease or disorder is a neurodegenerative disease or disorder; (c) the disease or disorder is ALS; (d) the disease or disorder is dementia; (e) the condition is ischemia; (f) the condition is a stroke; (g) the disease or disorder is AIDS; (h) the disease or disorder is multiple sclerosis; (i) the disease or disorder is Huntington's disease; (1) the disease is Parkinson's disease; (k) the disease or disorder is multiple organ failure; (l) the disease an inflammatory disease of immune and autoimmune origins; (m) the condition is tissues experiencing a physical or chemical insult; (n) the condition is shock; (o) the condition is skeletal muscle against damage; (p) the condition is myocardial tissue ischaemic damage; (q) the condition is neuronal tissue ischaemia damage; (r) the condition is donor tissue ischemia damage; (s) the patient is administered radiation prior to administration of the compound of Formula (I) formulation; (t) the patient is administered radiation after administration of the compound of Formula (I) formulation; (u) the patient is administered a different neuroprotective agent prior to administration of the compound of Formula (I) formulation; (v) the patient is administered a different neuroprotective agent after administration of the compound of Formula (I) formulation; (w) the diagnosis of the disease or disorder comprises administration of a compound of Formula (I); (x) the method further comprises whole brain radiation; (y) the method further comprises phototherapy; (z) the method further comprises assessment of the neurologic condition of the patient; (aa) the method further comprises administration of a zinc reagent; (ab) the method further comprises administration of an anti-inflammatory agent; (ac) the method further comprises administration of an anti-emetic agent; (ad) the method further comprises administration of a cytokine; (ae) the compound of Formula (I) localizes within a neuron; (af) the compound of Formula (I) localizes within a brain cell; (ag) the compound of Formula (I) localizes within a CNS cell; (ah) the compound of Formula (I) undergoes intracellular apical ligand exchange; (ai) the compound of Formula (I) is coordinated by at least one apical ligand derived from hydrochloric acid, nitric acid, acetic acid, gluconic acid, glucoronic acid, cholic acid, deoxycholic acid, methylphosphonic acid, phenylphdsphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid,; and (ak) the method further comprises surgery. In further aspects are methods in which the method has at least two of the aforementioned steps or characteristics; in further aspects, at least three of the aforementioned steps or characteristics; in further aspects, at least four of the aforementioned steps or characteristics; and in further aspects, at least five of the aforementioned steps or characteristics.

The term “acyl” refers to moieties having the formula R-C(O)—, wherein such moieties include, but are not limited to HC(O)—, alkyl-C(O)—, substituted alkyl-C(O)—, amino-C(O)—, substituted amino-C(O)—, cycloalkyl-C(O)—, substituted cycloalkyl-C(O)—, cycloalkenyl-C(O)—, substituted cycloalkenyl-C(O)—, alkenyl-C(O)—, substituted alkenyl-C(O)—, aryl-C(O)—, substituted aryl-C(O)—, heteroaryl-C(O)—, substituted heteroaryl-C(O)—, heterocyclic-C(O)—, substituted heterocyclic-C(O)—; where alkyl, substituted alkyl, cycloalkyl, substituted cycloalkyl, cycloalkenyl, substituted cycloalkenyl, amino, substituted amino, aryl, substituted aryl, heteroaryl, substituted heteroaryl, heterocyclic and substituted heterocyclic are as defined herein. Aminoacyl groups are sometimes also referred to as amides.

The term “acyloxy” refers to moieties having the formula R-C(O)O—, wherein such moieties include, but are not limited to HC(O)—, alkyl-C(O)O—, substituted alkyl-C(O)O—, amino-C(O)O—, substituted amino-C(O)O—, cycloalkyl-C(O)O—, substituted cycloalkyl-C(O)O—, cycloalkenyl-C(O)O—, substituted cycloalkenyl-C(O)O—, alkenyl-C(O)O—, substituted alkenyl-C(O)O—, aryl-C(O)O—, substituted aryl-C(O)O—, heteroaryl-C(O)O—, substituted heteroaryl-C(O)O—, heterocyclic-C(O)O—, substituted heterocyclic-C(O)O—; where alkyl, substituted alkyl, cycloalkyl, substituted cycloalkyl, cycloalkenyl, substituted cycloalkenyl, amino, substituted amino, aryl, substituted aryl, heteroaryl, substituted heteroaryl, heterocyclic and substituted heterocyclic are as defined herein.

The term “alkaryl” refers to the groups-optionally substituted alkylene-optionally substituted aryl, where alkylene, substituted alkylene, aryl and substituted aryl are defined herein. Such alkaryl groups are exemplified by benzyl, phenethyl and the like.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), 1-propylene (—CH₂CH═CH₂), isopropylene [—C(CH₃)═CH₂], and the like.

The term “substituted alkenyl” refers to an alkenyl group in which at least 1 hydrogen atoms has been replaced by a substituent selected from ═O, ═S, acyl, acyloxy, alkoxy, substituted alkoxy, amino, substituted amino, aryl, substituted aryl, aryloxy, substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, halogen, hydroxyl, nitro, phosphine, phosphonato, phosphono, sulfanyl, sulfinyl, sulfonyl, substituted phosphine, substituted phosphonato, substituted phosphono, substituted sulfanyl, substituted sulfinyl, substituted sulfonyl, cycloalkyl, substituted cycloalkyl, cycloalkenyl, substituted cycloalkenyl, acylamino, acyloxy, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, substituted heteroaryl, heteroaryloxy, substituted heteroaryloxy, heterocyclyl, substituted heterocyclyl, heterocyclooxy, substituted heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkenylene” refers to a diradical derived from the above-defined monoradical, alkenyl. This term is exemplified by groups such as ethenylene (—CH═CH—), the propenylene isomers (e.g., —CH₂CH═CH— and —C(CH₃)═CH—) and the like.

The term “substituted alkenylene” refers to a diradical derived from the above-defined monoradical, substituted alkenyl.

The term “alkoxy” refers to moieties having the formula —O—R, wherein such moieties include, but are not limited to, —O-alkyl, —O-alkenyl, —O-cycloalkyl, —O-cycloalkenyl, —O-alkynyl. In addition, non-limiting examples of such —O-alkyl groups are methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to moieties having the formula —O-(substituted alkyl), —O-(substituted alkenyl), —O-(substituted cycloalkyl), —O-(substituted cycloalkenyl), —O-(substituted alkynyl), —O-(substituted alkylene)-alkoxy. Non-limiting examples of such —O-(substituted alkylene)-alkoxy, also referred to as “polyalkoxy”, are —OCH₂CH₂OCH₃, and polyethylene glycol (PEG) groups such as —O(CH₂CH₂O)ₙCH₃, where x is an integer of about 1-20. Non-limiting examples of such —O-(substituted alkyl) groups are —OCH₂(CH₂)_{y}OH, where y is an integer of about 1-10, preferably about 14.

The term “alkoxyalkylene” refers to the groups: alkyl-O-alkylene-, (substituted alkyl)-O-alkylene-, alkyl-O-substituted alkylene-, (substituted alkyl)-O-(substituted alkylene). A non-limiting examples of such alkoxyalkylene group is -alkylene-O-alkyl and include, by way of example, methoxymethylene (—CH₂OCH₃), methoxyethylene (—CH₂CH₂OCH₃), n-(iso-propoxy)propylene [—CH₂CH₂CH₂OCH(CH₃)₂] and the like.

The term “alkyl” refers to a monoradical branched, cyclic, or unbranched saturated hydrocarbon chain preferably having from 1 to 20 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group as defined herein, having at least 1 substituent selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl; or an alkyl group as defined herein that is interrupted by 1-20 atoms independently chosen from oxygen, sulfur and NR^{a}—, where R^{a} is chosen from hydrogen, or optionally substituted alkyl, cycloalkyl, alkenyl, cycloalkenyl, alkenyl, cycloalkenyl, alkynyl, aryl, heteroaryl and heterocyclic; or an alkyl group as defined herein that has both from 1 to 5 substituents as defined herein and is also interrupted by 1-20 atoms as defined herein.

A non-limiting example of an alkyl substituent is hydroxy, exemplified by hydroxyalkyl groups, including but not limited to, 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and the like; and those compounds known as polyethylene glycols, polypropylene glycols and polybutylene glycols, and the like.

The term “alkylene” refers to a diradical of a branched, cyclic, or unbranched saturated hydrocarbon chain, preferably having from 1 to 20 carbon atoms, preferably 1-10 carbon atoms, more preferably 1-6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene” refers to an alkylene group as defined herein having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyacylamino, azido, cyano, halogen, hydroxyl, keto, thioketo, carboxyl, carboxylalkyl, thiol, thioalkoxy, substituted thioalkoxy, aryl, aryloxy, thioaryloxy, heteroaryl, heteroaryloxy, thioheteroaryloxy, heterocyclic, heterocyclooxy, thioheterocyclooxy, nitro, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Additionally, such substituted alkylene groups include those where two substituents on the alkylene group are fused to form one or more cycloalkyl, substituted cycloalkyl, cycloalkenyl, substituted cycloalkenyl, aryl, heterocyclic or heteroaryl groups fused to the alkylene group; or an alkylene group as defined herein that is interrupted by 1-20 atoms independently chosen from oxygen, sulfur and NR^{a}—, where R^{a} is chosen from hydrogen, optionally substituted alkyl, cycloalkyl, alkenyl, cycloalkenyl, alkenyl, cycloalkenyl, alkynyl, aryl, heteroaryl and heterocyclic, or groups selected from carbonyl, carboxyester, carboxyamide and sulfonyl; or an alkylene group as defined herein that has both from 1 to 5 substituents as defined herein and is also interrupted by 1-20 atoms as defined herein.

Examples of substituted alkylenes are chloromethylene (—CH(Cl)—), aminoethylene (—CH(NH₂)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein. Alkylthioalkoxy groups include alkylene-S-alkyl, by way of example, methylenethiomethoxy (—CH₂SCH₃), ethylenethiomethoxy (—CH₂CH₂SCH₃), n-propylene-iso-thiopropoxy (—CH₂CH₂CH₂SCH(CH₃)₂), methylene-t-thiobutoxy (—CH₂SC(CH₃)₃) and the like.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl, (—C≡CCH₃), and the like.

The term “substituted alkynyl” refers to an alkynyl group as defined herein having from 1 to 5 substituents, and preferably 1 to 3 substituents, selected from the group consisting of: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, phosphono, sulfanyl, sulfinyl, and sulfonyl.

The term “alkynylene” refers to a diradical of an unsaturated hydrocarbon preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynylene groups include ethynylene (—C═C—), propargylene (—CH₂—C≡C—) and the like.

The term “substituted alkynylene” refers to an alkynylene group as defined herein having from 1 to 5 substituents, and preferably 1 to 3 substituents, selected from the group consisting of: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, phosphono, sulfanyl, sulfinyl, and sulfonyl.

The term “acylamino” or “aminocarbonyl” refers to the group —C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl and heterocyclic are as defined herein.

The term “amino” refers to the group —NH₂.

The term “substituted amino” refers to the group —NHR or —NRR where each R is independently selected from the group: acyl, optionally substituted alkenyl, optionally substituted alkyl, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, and optionally substituted heterocyclyl. Preferred amino substituents include optionally substituted alkyl, aryl, optionally substituted alkoxycarbonyl, optionally substituted aminocarbonyl, and heteroaryl.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” or “alkoxycarbonylamino” refers to the group —NRC(O)OR where each R is independently hydrogen, alkyl, substituted alkyl, aryl, heteroaryl, or heterocyclic wherein alkyl, substituted alkyl, aryl, heteroaryl and heterocyclic are as defined herein.

The term “apical ligand” refers to an anion that binds to the core metal of the metallotexaphyrin, e.g., with de-localized electrostatic or weak coordinate-covalent bonds. The number of apical ligands (n) is defined as an integer of 1-5. It should be noted that the apical ligands act to neutralize the charge on the metallotexaphyrin. Thus, typically n is 1 when M is a divalent cation, and n is 2 when M is a trivalent cation (because the core itself neutralizes one unit charge). However, if any of R¹, R^{1′}, R², R³, R⁴, R⁴′, R⁵, R⁶, R⁷, R⁸, R⁹, R¹⁰, R¹¹ and R¹² is capable of forming an acid addition salt, for example a carboxylate or a phosphate, then n can decrease appropriately. It is also possible that the apical ligands could have two functionalities capable of forming an anion, for example a dicarboxylic acid, and such ligands are intended to be within the scope of the invention. In general, any molecule containing a carboxylic acid or phosphate may be used as an apical ligand, for example biomolecules, including lipoproteins, estradiol and amino acids, carboxylates of sugar derivatives, such as gluconic acid or glucoronic acid, cholesterol derivatives such as cholic acid and deoxycholic acid, PEG acids, organophosphates, such as methylphosphonic acid and phenylphosphonic acid, and phosphoric acid or other inorganic acids, and the like, or sulfonic acid derivatives such as methanesulfonic acid, ethanesulfonic acid or “carboxylic acid derivatives”, which term refers to compounds of the formula R-CO₂H, in which R is optionally substituted alkyl, optionally substituted alkenyl, optionally substituted alkynyl, or optionally substituted aryl, as defined above. Preferred are gluconic and glucuronic acid, and those carboxylic acid derivatives where R is optionally substituted alkyl, for example acids of 1-20 carbon atoms, such as formic acid, acetic acid, propionic acid, butyric acid, pentanoic acid, 3,6,9-trioxodecanoic acid, 3,6-dioxoheptanoic acid, methylvaleric acid, glycolic acid, pyruvic acid, oxalic acid, malic acid, malonic acid, succinic acid, maleic acid, fumaric acid, tartaric acid, citric acid, and the like. Also preferred are those carboxylic acid derivatives where R is aryl, in particular where R is optionally substituted phenyl, for example benzoic acid, salicylic acid, 3-fluorobenzoic acid, 4-aminobenzoic acid, cinnamic acid, mandelic acid, p-toluene-sulfonic acid, and the like. Other examples of apical ligands include: OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, and OCN⁻.

The term “aromatic” refers to a cyclic or polycyclic moiety having a conjugated unsaturated (4n+2) π electron system (where n is a positive integer), sometimes referred to as a delocalized it electron system.

The term “aryl” refers to an unsaturated aromatic carbocyclic group of from 6 to 20 carbon atoms having a single ring (e.g., phenyl) or multiple condensed (fused) rings (e.g., naphthyl or anthryl). Preferred aryls include phenyl, naphthyl and the like.

Unless otherwise constrained by the definition for the aryl substituent, such aryl groups can optionally be substituted with from 1 to 5 substituents, preferably 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂substituted alkyl, —SO₂-aryl, —SO₂-heteroaryl and trihalomethyl. Preferred aryl substituents include alkyl, alkoxy, halo, cyano, nitro, trihalomethyl, and thioalkoxy.

The term “aralkyl” refers to the moiety “-arylene-alkyl,” each subpart having the meaning as defined herein.

The term “substituted aralkyl” refers to the moiety “-(optionally substituted arylene)-(optionally substituted alkyl)”, each having the meaning as defined herein.

The term “aryloxy” refers to the group aryl-O— wherein the aryl group is as defined herein including optionally substituted aryl groups as also defined herein.

The term “arylene” refers to the diradical derived from aryl (including substituted aryl) as defined herein and is exemplified by 1,2-phenylene, 1,3-phenylene, 1,4-phenylene, 1,2-naphthylene and the like.

The term “carbonyl” refers to the di-radical “—C(═O)—” which is also written as “—C(O)—”.

The term “(optionally substituted alkoxy)carbonyl” refers to the groups: —C(O)O-(optionally substituted alkyl), —C(O)O-(optionally substituted cycloalkyl), —C(O)O-(optionally substituted alkenyl), and —C(O)O-(optionally substituted alkynyl). These moieties are also referred to as esters, carboxylalkyls or alkoxycarbonyls.

The term “(optionally substituted amino)carbonyl” refers to the group —C(O)-(optionally substituted amino). This moiety is also referred to as an amide, or a primary, secondary or tertiary carboxamide.

The term “(optionally substituted alkyl)carbonyloxy” refers to the group —O—C(O)-(optionally substituted alkyl).

The term “(optionally substituted amino)carbonyloxy” refers to the group —O—C(O)-(optionally substituted amino).

The term “carboxy” or “carboxyl” refers to the moiety “—C(O)OH”, which is also illustrated as “—COOH”.

The term “catalytic group” means a chemical functional group that assists catalysis by acting as a general acid, Brønsted acid, general base, Brønsted base, nucleophile, or any other means by which the activation barrier to reaction is lowered. Exemplary catalytic groups contemplated include, but are not limited to, imidazole; guanidine; substituted saccharides such as D-glucosamine, D-mannosamine, D-galactosamine, D-glucamine and the like; amino acids such as L-histidine and L-arginine; derivatives of amino acids such as histamine; polymers of amino acids such as poly-L-lysine, (LysAla), (LysLeuAla)ₙ where n is from 1-30 or preferably 1-10 or more preferably 2-7 and the like; derivatives thereof; and metallotexaphyrin complexes.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “cycloalkylene” refers to the diradical derived from cycloalkyl as defined herein and is exemplified by 1,1-cyclopropylene, 1,2-cyclobutylene, 1,4-cyclohexylene and the like.

The term “substituted cycloalkyl” refers to cycloalkyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkylene” refers to the diradical derived from substituted cycloalkyl as defined herein.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl and the like.

The term “cycloalkenylene” refers to the diradical derived from cycloalkenyl as defined herein and is exemplified by 1,2-cyclobut-1-enylene, 1,4-cyclohex-2-enylene and the like.

The term “substituted cycloalkenyl” refers to cycloalkenyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkenylene” refers to the diradical derived from substituted cycloalkenyl as defined herein.

The term “halo” or “halogen” refers to fluoro, chloro, bromo and iodo.

The term “heteroaryl” refers to an aromatic group comprising 1 to 15 carbon atoms and 1 to 4 heteroatoms selected from oxygen, nitrogen and sulfur within at least one ring (if there is more than one ring).

Unless otherwise constrained by the definition for the heteroaryl substituent, such heteroaryl groups can be optionally substituted with 1 to 5 substituents, preferably 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “heteroaryloxy” refers to the group heteroaryl-O—.

The term “heteroarylene” refers to the diradical group derived from heteroaryl (including substituted heteroaryl), as defined herein, and is exemplified by the groups 2,6-pyridylene, 2,4-pyridiylene, 1,2-quinolinylene, 1,8-quinolinylene, 1,4-benzofuranylene, 2,5-pyridnylene, 2,5-indolenyl and the like.

The term “heterocycle” or “heterocyclic” refers to a monoradical saturated or unsaturated group having a single ring or multiple condensed rings, having from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Such heterocyclic groups can have a single ring or multiple condensed rings. Preferred heterocyclics include morpholino, piperidinyl, and the like.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

The term “heterocyclooxy” refers to the group heterocyclic-O—.

The term “heterocyclene” refers to the diradical group formed from a heterocycle, as defined herein, and is exemplified by the groups 2,6-morpholino, 2,5-morpholino and the like.

The term “linker” as used herein means a covalent connection of a functional group (e.g., a site directing group, a catalytic group or a neuroprotective agent) to a metallotexaphyrin, and may be, for example, a covalent bond or an alkylene, alkenylene, alkynylene, arylene, ethers, PEG moieties, and the like, all of which may be optionally substituted. Examples of reactions to form a covalent link include reaction between an amine (on either the functional group or the linker precursor) with a carboxylic acid (on the other) to form an amide link. Similar reactions well known in the art are described in standard organic chemistry texts such as J. March, “Advanced Organic Chemistry”, 4^{th} Edition, (Wiley-Interscience (New York), 1992.

The term “phosphate” refers to the group —O—PO₃H₂. One or more of the hydrogen atoms on the phosphate group may be substituted with alkyl, alkenyl, alkynyl, aryl, heteroaryl, or heterocycyle.

The term “phosphine” refers to the group —PH₂.

The term “substituted phosphine” refers to the group —PR′R″ where R′ and R″ are selected from the group: hydrogen, alkyl, alkoxy and aryl, and at least one of R′ or R″ is not hydrogen.

The term “phosphonato” refers to the group —P(O)(O⁻)₂, which, depending upon whether one or more of the oxygen anions is linked to another moiety (such as ribose, in the case of RNA) is sometimes also referred to as a phosphodiester linkage.

The term “phosphono” refers to the group —P(O)(OH)₂, which is sometimes also referred to as a phosphate. One or more of the hydrogen atoms on the phosphate group may be substituted with alkyl, alkenyl, alkynyl, aryl, heteroaryl, or heterocycyle.

The term “site-directing group” refers to a functional group having an affinity for a biological receptor or for a nucleic acid sequence. Exemplary site-directing groups useful herein include, but are not limited to, polydeoxyribonucleotides, oligodeoxyribonucleotides, polyribonucleotide analogs, oligoribonucleotide analogs, polyamides including peptides having affinity for a biological receptor and proteins such as antibodies, steroids and steroid derivatives, hormones such as estradiol or histamine, hormone mimics such as morphine, and further macrocycles such as sapphyrins and rubyrins. The oligonucleotides may be derivatized at the bases, the sugars, the ends of the chains, or at the phosphate groups of the backbone to promote in vivo stability. Modifications of the phosphate groups are preferred in one embodiment since phosphate linkages are sensitive to nuclease activity. Presently preferred derivatives are the methylphosphonates, phosphotriesters, phosphorothioates, and phosphoramidates. Additionally, the phosphate linkages may be completely substituted with non-phosphate linkages such as amide linkages. Appendages to the ends of the oligonucleotide chains also provide exonuclease resistance. Sugar modifications may include groups, such as halo, alkyl, alkenyl or alkoxy groups, attached to an oxygen of a ribose moiety in a ribonucleotide. In a preferred embodiment, the group will be attached to the 2′ oxygen of the ribose. In particular, halogen moieties such as fluoro may be used. The alkoxy group may be methoxy, ethoxy or propoxy. The alkenyl group is preferably allyl. The alkyl group is preferably a methyl group and the methyl group is attached to the 2′ oxygen of the ribose. Other alkyl groups may be ethyl or propyl. It is understood that the terms “nucleotide”, “polynucleotide” and “oligonucleotide”, as used herein, refer to both naturally-occurring and synthetic nucleotides, poly- and oligonucleotides and to analogs and derivatives thereof such as methylphosphonates, phosphotriesters, phosphorothioates, phosphoramidates and the like. Deoxyribonucleotides, deoxyribonucleotide analogs and ribonucleotide analogs are contemplated as site-directing groups in the present invention. The term “texaphyrin-oligonucleotide conjugate” means that an oligonucleotide is attached to the texaphyrin in a 5′ or a 3′ linkage, or in both types of linkages to allow the texaphyrin to be an internal residue in the conjugate. It can also refer to a texaphyrin that is linked to an internal base of the oligonucleotide. The oligonucleotide or other site-directing group may be attached either directly to the texaphyrin or to the texaphyrin via a linker or a couple of variable length.

The term “sulfanyl” refers to the groups: —S-(optionally substituted alkyl), —S-(optionally substituted aryl), —S-(optionally substituted heteroaryl), —S-(optionally substituted heterocyclyl). Preferred sulfanyl groups include, by way of example, methylsulfanyl (—SCH₃), n-(iso-propylsulfanyl) (—SCH(CH₃)₂) and the like.

The term “sulfinyl” refers to the groups: —S(O)-(optionally substituted alkyl), —S(O)-optionally substituted aryl), —S(O)-(optionally substituted heteroaryl), —S(O)-(optionally substituted heterocyclyl). Preferred sulfinyl groups include, by way of example, methylsulfinyl (—S(O)—CH₃) and the like.

The term “sulfonyl” refers to the groups: —S(O₂)-(optionally substituted alkyl), —S(O₂)-optionally substituted aryl), —S(O₂)-(optionally substituted heteroaryl), —S(O₂)-(optionally substituted heterocyclyl). Preferred sulfonyl groups include, by way of example, methylsulfonyl (—S(O₂)—CH₃) and the like.

“Texaphyrin” means an aromatic pentadentate macrocyclic expanded porphyrins, also described as an aromatic benzannulene containing both 18π- and 22π-electron delocalization pathways. Texaphyrins and water-soluble texaphyrins, methods of preparation and various uses and the like have been described, for example, in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,530,122, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, 5,714,328, 5,733,903, 5,744,302, 5,756,726, 5,763,172, 5,775,339, 5,776,925, 5,798,491, 5,801,229, 5,808,059, 5,817,017, 5,837,866, 5,886,173, 5,888,997, 5,955,586, 5,969,111, 5,994,935, 6,022,526, 6,022,959, 6,069,140, 6,072,038, 6,096,030, 6,207,660, 6,270,749, 6,375,930, 6,638,924, 6,657,058, 6,825,186, 6,919,327, in PCT publications WO 90/10633, 94/29316, 95/10307, 95/21845, 96/09315, 96/40253,96/38461, 97/26915, 97/35617, 97/46262, 98/07733, 98/25648, 99/09411, 99/15236, 99/62551, 00/01413, 00/01414, 03/37888; 05/112759; and in pending U.S. patent application Ser. Nos. 10/160,205, 10/659,499, 10/310,592, 10/363,401, 10/362,964, 10/318, 659, 10/911,284, 11/241,549, 11/235,475, and 60/737,601, each of which are herein incorporated by reference in their entirety.

The term “spiro-attached cycloalkyl group” refers to a cycloalkyl group attached to another ring via one carbon atom common to both rings.

The term “thiol” refers to the group —SH.

The term “thioalkoxy” refers to the group —S-alkyl.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclic-S—.

The term “saccharide” includes oxidized, reduced or substituted saccharides, including hexoses such as D-glucose, D-mannose or D-galactose; pentoses such as D-ribose or D-arabinose; ketoses such as D-ribulose or D-fructose; disaccharides such as sucrose, lactose, or maltose; derivatives such as acetals, amines, and phosphorylated sugars; oligosaccharides; as well as open chain forms of sugars, and the like. Examples of amine-derivatized sugars are galactosamine, glucosamine, and sialic acid.

The term “optionally substituted polyether” refers to any group of the formula O-(alkylene-O)ₙ-alkyl, where n is a number between 1 and 100, preferably 1 and 10, and wherein the alkylene and alkyl groups are optionally substituted as defined herein.

The term “substituted hydroxylated group” refers to any chemical group defined herein in which one or more —OH groups are present; further substituents on such a hydroxylated group are permitted as defined herein for each chemical group. Preferably a hydroxylated group contains at least two —OH groups.

The term “parenteral administration” as described herein, refers to administration of at least one agent by means other than through the alimentary tract. Parenteral routes of administration involve injections into various compartments of the body such as but not limited to, intravenous, subcutaneous, intramuscular, intraperitoneal and the like.

The terms “pharmaceutically effective amount” or “effective amount” as described herein, refers to a nontoxic but sufficient amount of the agent to provide the desired biological, therapeutic, and/or prophylactic result. The desired results include reduction and/or alleviation of the signs, symptoms, or causes of a disease, or any other desired alteration of a biological system. An effective amount in any individual case may be determined by one of ordinary skill in the art using routine experimentation.

The term “pharmaceutically acceptable” or “pharmacologically acceptable” as described herein, may mean a material which is not biologically or otherwise undesirable, i.e., the material may be administered to an individual without causing any undesirable biological effects or interacting in a deleterious manner with any of the components of the composition in which it is contained.

As used herein, “pharmaceutically acceptable carrier” or “pharmaceutically acceptable excipient” includes any and all non-active components of a pharmaceutical composition, including by way of example only solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalcyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “photodynamic therapy” as described herein, refers to a treatment that combines a light source and a photosensitizing agent (a drug that is activated by light).

The term “radiation therapy” as described herein, refers to exposing a patient to high-energy radiation, including without limitation x-rays, gamma rays, and neutrons. This type of therapy includes without limitation external-beam therapy, internal radiation therapy, implant radiation, brachytherapy, systemic radiation therapy, and radiotherapy.

The term “same molecular weight,” as used herein, refers to compounds that have the same molecular weight, excluding isotopic variations and the identity of the counterions. That is, the molecular weight of a compound is determined by adding up the atomic weight of all atoms in the formula, excluding however, the counterions (i.e., the X or AL groups in Formula (I)). In particular, a compound does not have a different molecular weight from another compound, for purposes of this definition because it has a ²H instead of a ¹H in a structure (i.e., isotopic variations do not constitute different molecular weights).

The term “surgery” as described herein, refers to any therapeutic or diagnostic procedure that involves methodical action of the hand or of the hand with an instrument, on the body of a human or other mammal, to produce a curative, remedial, or diagnostic effect.

The term “treating” and its grammatical equivalents as described herein, refers to achieving, or attempting to achieve, a therapeutic benefit and/or a prophylactic benefit. By therapeutic benefit is meant eradication or amelioration, at least in part, of the underlying disorder being treated. For example, in a patient with a neurologic condition, therapeutic benefit includes eradication or amelioration, at least in part, of the underlying neurologic condition. Also, a therapeutic benefit includes the eradication or amelioration, at least in part, of one or more of the physiological symptoms associated with the underlying disorder such that an improvement is observed in the patient, notwithstanding the fact that the patient may still be afflicted with the underlying disorder. For prophylactic benefit, a method disclosed herein may be performed on, or a composition disclosed herein administered to, a patient at risk of developing a neurologic condition, or to a patient reporting one or more of the physiological symptoms of such conditions, even in the absence of a diagnosis of the condition.

The term “therapeutically effective amount” refers to that amount of a compound of Formula (I) that is sufficient to effect treatment, as defined below, when administered to a mammal in need of such treatment. The therapeutically effective amount will vary depending upon the subject and disease condition being treated, the weight and age of the subject, the severity of the disease condition, the particular compound of Formula (I) chosen, the dosing regimen to be followed, timing of administration, the manner of administration and the like, all of which can readily be determined by one of ordinary skill in the art.

As to any of the above groups that contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

### INCORPORATION BY REFERENCE

Unless stated otherwise, all publications and patent applications mentioned in this specification are herein incorporated by reference to the same extent as if each individual publication or patent application was specifically and individually indicated to be incorporated by reference.

### DETAILED DESCRIPTION OF THE INVENTION

Texaphyrins

Expanded porphyrins metal complexes, including the texaphyrin metal complexes described herein, can be used for the treatment of a variety of neurodegenerative diseases and disorders and/or free-radical associated diseases and disorders. Such neurodegenerative diseases and disorders, also referred to as neurologic diseases and disorders, include but are not limited to, amyotrophic lateral sclerosis (ALS or Lou Gerhig's Disease), Alzheimer's disease, multiple sclerosis, dementia, AIDS dementia, Parkinson's disease, motor neuron disorders, and Huntington's disease. The texaphyrin metal complexes described herein, which may be used as neuroprotective agents (prophylactics) and/or to treat such neurodegenerative diseases and disorders and/or free-radical associated diseases and disorders have the structure of Formula (I), 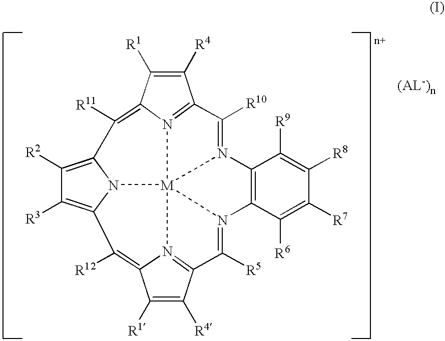
wherein:
M is a transition metal ion or a lanthanide metal ion,
AL is an apical ligand;
n is 1,2,3,4, or 5;
R⁶ and R⁹ are independently chosen from the group: acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, sulfanyl, sulfinyl, sulfonyl, and the moiety —X-Y where: X is a covalent bond or a linker, and Y is a catalytic group, a neuroprotectiv agent or a site-directing group;
R¹, R^{1′}, R², R³, R⁴, R⁴′, R⁷ and R⁸ are independently chosen from the group: acyl, acyloxy, alkyl, optionally substituted alkenyl, optionally substituted alkoxy, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, sulfanyl, sulfinyl, sulfonyl, and the moiety —X-Y where: X is a covalent bond or a linker, and Y is a catalytic group, a neuroprotective agent or a site-directing group; and
R⁵, R¹⁰, R¹¹ and R¹² are independently chosen from the group: acyl, optionally substituted alkoxy, optionally substituted alkyl, optionally substituted aryl, halo, and hydrogen;
   with the proviso that for R⁶ and R⁹, halogen is other than iodide and substituted alkyl is other than iodoalkyl; and with the proviso that at least one of R¹, R^{1′}, R², R³, R⁴, R⁴′, R⁷ and R⁸ is —O-(optionally substituted alkylene-O)ₙ-alkyl, where n is 1, 2, 3, 4, 5, 6, 7, 8, 9, or 10.

In certain embodiments when Y is a neuroprotective agent, Y may be selected from antioxidants, glutamate antagonists, metal chelators, neural growth factors, non-neural growth factors, calcium regulators, anti-inflammatory agents, inhibitors of cell signaling pathways, inhibitors of cell death pathways, dietary supplements, energetic precursors (by way of example creatine), immunoregulatory agents, chloinergic agents, dopaminergic agents and anti-viral agents.

In certain embodiments M of Formula (I) is selected from the group consisting of lanthanum, cerium, praseodynmium, neodynium, promethium, samarium, europium, gadolinium, terbium, dysprosium, holmium, erbium, thulium, ytterbium and lutetium.

In certain embodiments M of Formula (I) is selected from titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, ununnilium, unununium, or ununbium.

In certain embodiments M of Formula (I) is selected from Mn⁺, Mn³⁺, Mn⁴⁺, Mn⁷⁺, Co²⁺, Co³⁺, Ni²⁺, Ni³⁺, Zn²⁺, Cd²⁺ Hg²⁺, Fe²⁺, Sm²⁺, U²⁺, Mn³⁺, Fe³⁺, CuI+, Cu²⁺, Ho³⁺, Ce³⁺, Ce⁴⁺, Y³⁺, In³⁺, Pr ³⁺, Nd ³⁺, Sm³⁺, Eu²⁺, Eu³⁺, Ru²⁺, Ru³⁺, Re⁴⁺, Re⁶⁺, Re⁷⁺, Gd³⁺, Tb3+, Tc⁴⁺, Tc⁶⁺, Tc⁷⁺, Dy³⁺, Er³⁺, Tm³⁺, Yb²⁺, Yb³⁺, Lu³⁺, La³⁺, U³⁺, OS³⁺, OS⁴⁺, or other cations of the lanthanide series.

In certain embodiments each AL is independently selected from chloride, nitrate, acetate, and hydroxide, or are formed from carboxylates of sugar derivatives, such as gluconic acid or glucoronic acid, cholesterol derivatives such as cholate and deoxycholate, or derivatives of PEG acids, organic acids such as formic acid, acetic acid, propionic acid, butyric acid, pentanoic acid, methylvaleric acid, glycolic acid, pyruvic acid, oxalic acid, malic acid, malonic acid, succinic acid, maleic acid, fumaric acid, tartaric acid, benzoic acid, 3,6,9-trioxodecanoic acid, 3,6-dioxoheptanoic acid, 3,6-dioxoheptanoic acid, 2,5-dioxoheptanoic acid, citric acid, benzoic acid, cinnamic acid, mandelic acid, salicylic acid, methanesulfonic acid, ethanesulfonic acid, p-toluene-sulfonic acid, organophosphates, such as methylphosphonic acid and phenylphosphonic acid, phosphoric acid, pyridine, benzimidazole, methanol, water, or inorganic acids, and the like.

Non-limiting examples of such texaphyrin metal complexes of Formula (I) includes the texaphyrins having the structure of Formula (II), Formula (III), Formula (IV), and Formula (V):

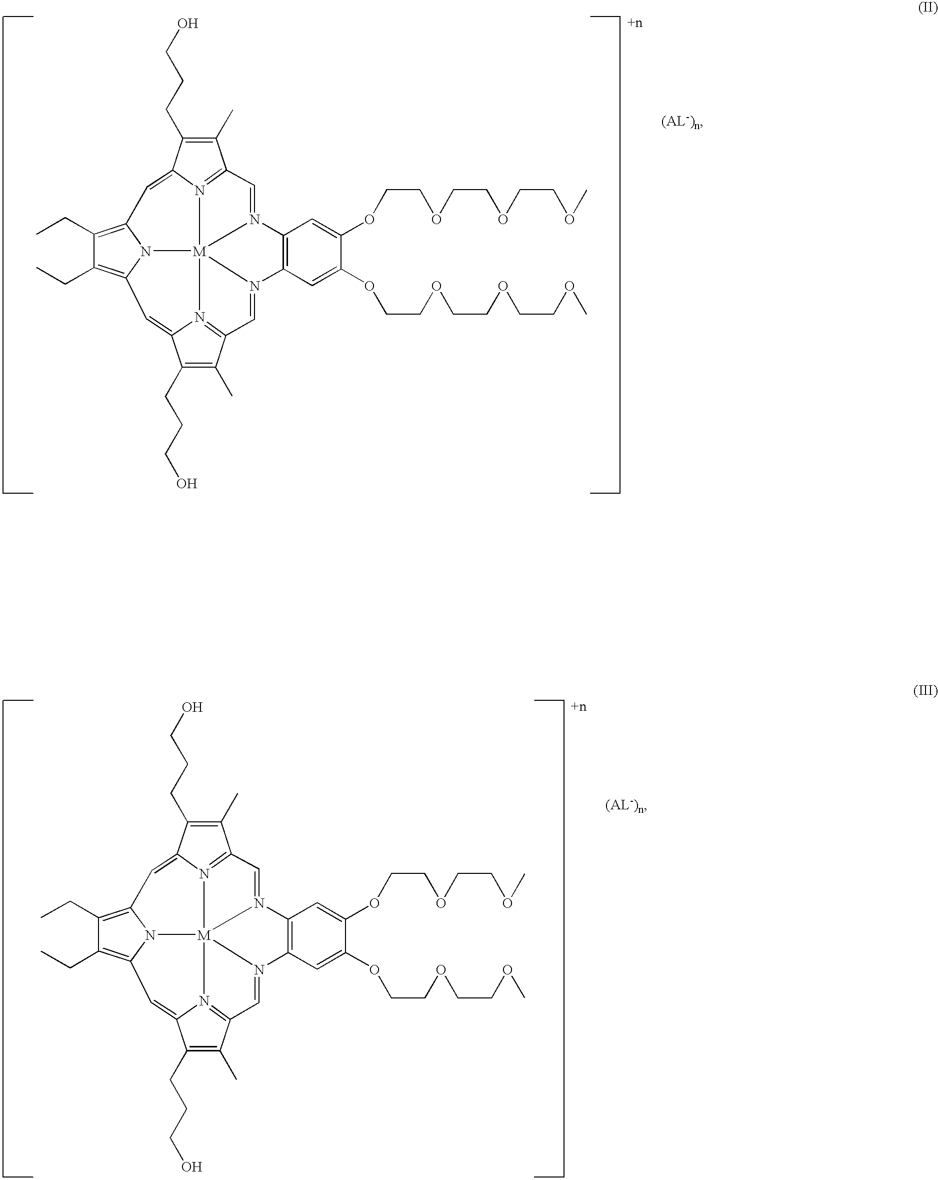

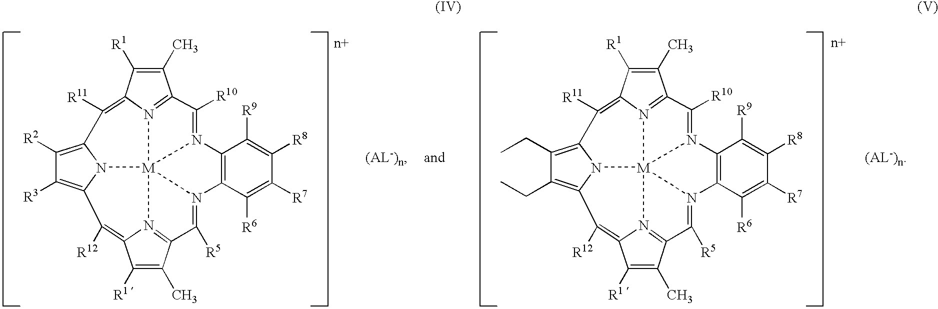

Non-limiting examples of such texaphyrins metal complexes of Formula (I) includes the texaphyrins having the following structures of

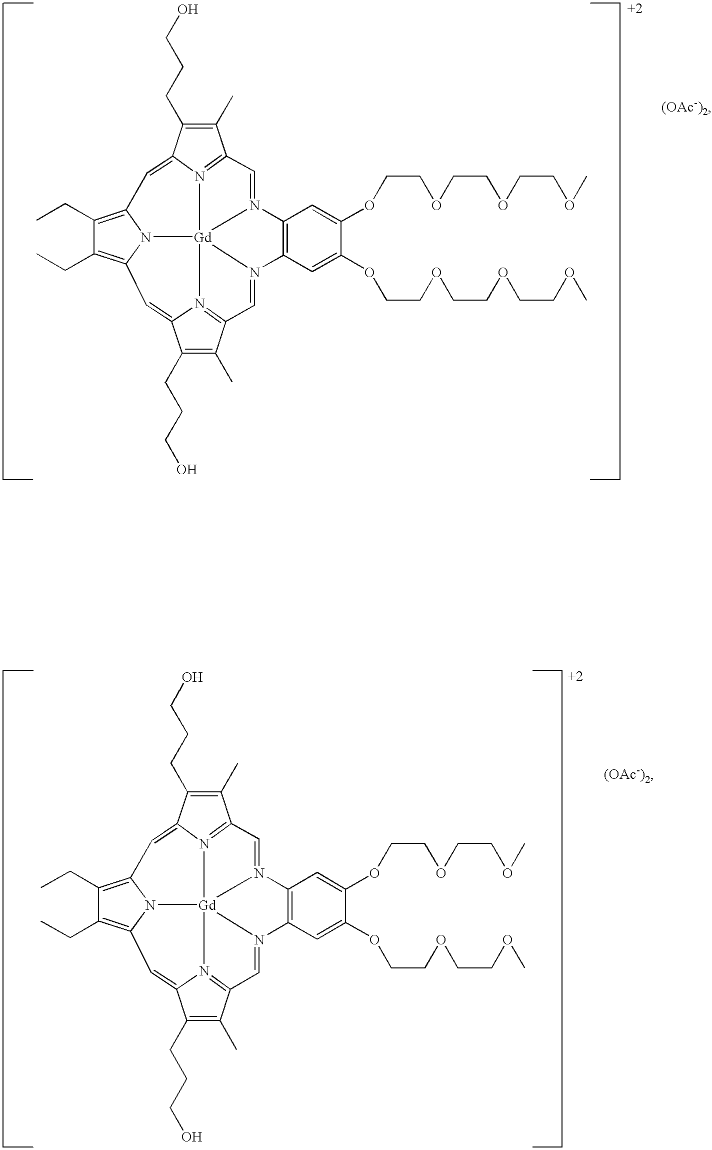

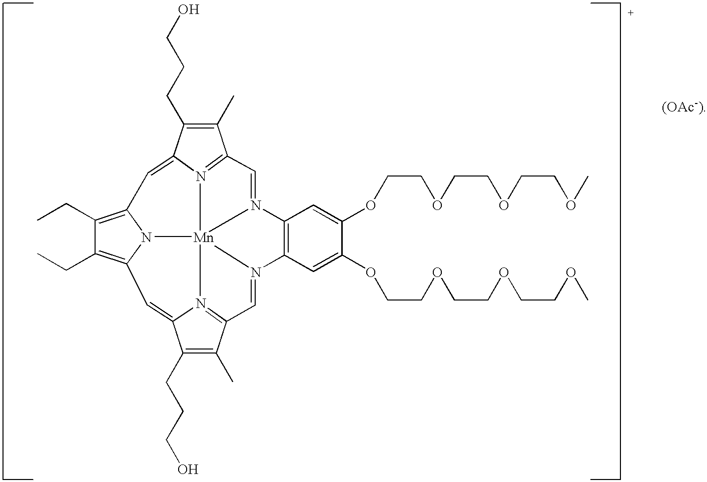

The incorporation of hydroxylated groups and/or polyether groups into such texaphyrins may allow for the modification of the therapeutic index of such texaphyrins. By way of example only, such hydroxylated groups include, but are not limited to, sugars, carbohydrates, and saccharides. Additionally, by way of example only, such polyether groups include, but are not limited to, polyethylene glycol.

In certain embodiments, compounds of Formula (I) are high purity texaphyrins, wherein at least about 95% of compounds of Formula (I) have the same structure, the same molecular weight, excluding isotopic variation, and wherein both polyethylene glycol chain lengths on the aromatic moiety have the same chain length. In other embodiments disclosed herein, at least about 98.7%, 99%, 99.3% or 99.5% of the compounds of Formula (I) in the high purity sample have the same structure, the same molecular weight, excluding isotopic variation, and wherein both polyethylene glycol chain lengths on the aromatic moiety have the same chain length. In other embodiments such high purity texaphyrins of Formula (I) have less than about 1.6% polydispersity of the hydroxylated groups on the aromatic moiety. In further embodiments such high purity texaphyrins of Formula (I) have less than about 1.6% polydispersity of the polyether chain on the aromatic moiety. In further embodiments such high purity texaphyrins of Formula (I) have less than about 1.6% polydispersity of the polyethylene glycol chain on the aromatic moiety. Such high purity texaphyrins are synthesized, purified and analyzed using the methods and techniques described in U.S. patent application Ser. No. 11/235,475, which is herein incorporated by reference in its entirety.

Without limiting the scope of the compositions and methods disclosed herein, some of the methods for demonstrating a purity of a compound include, but are not limited to:(i) chromatographic methods, by way of example only, molecular size exclusion chromatography, native gel electrophoresis, high pressure liquid chromatography (HPLC), liquid chromatography (LC), liquid chromatography coupled with mass spectroscopy (LC/MS), gas chromatography (GC), GC coupled with mass spectroscopy (GC MS), supercritical fluid chromatography, gel permeation chromatography and ion exchange chromatography, and Reversed-Phase High Performance Liquid Chromatography; (ii) end group analysis; (iii) vapor pressure osmometry; (iv) cryoscopy/ebulliometry, by way of example only, freezing point depression/boiling point elevation; (v) viscometry; (vi) small-angle X ray scattering; (vii) laser light scattering; (viii) optical absorption and scattering; (ix) ultracentrifugation; (x) field flow fractionation; (xi) matrix-assisted laser desorption/ionization time-of-flight (MALDI-TOF) mass spectrometry; (xii) nuclear magnetic resonance spectrometry, and (xiii) crystallization.

Methods for Treating Neurodegenerative Diseases and Disorders and/or Free-Radical Associated Diseases and Disorders

For convenience, the methods and compositions for treating neurodegenerative diseases and disorders and/or free-radical associated diseases and disorders described in this section have been described generically and/or with specific examples. However, the methods and compositions for treating neurodegenerative diseases and disorders described in this section should not be limited to just the generic descriptions or specific example provided in this section, but rather the methods and compositions for treating neurodegenerative diseases and disorders described in this section apply equally well to all compounds that fall within the scope of Formulas I-V, including any sub-formulas or specific compounds that fall within the scope of Formulas I-V that are described in the specification, claims and figures herein.

Without limiting the scope of the compositions and the methods disclosed herein, the compositions and methods described herein are used as neuroprotective agents (prophylactics) and/or to treat several neurodegenerative diseases and disorders, including but not limited to, amyotrophic lateral sclerosis (ALS or Lou Gerhig's Disease), Alzheimer's disease, dementia, AIDS dementia, Parkinson's disease, motor neuron disorders, stroke, and Huntington's disease. In addition, the compositions and methods described herein are used to treat free-radical associated diseases and disorders, including but not limited to, chronic inflammation, arthritis, autoimmune diseases, ischemia-reperfusion injury, septic shock and chronic graft rejection. In addition, the compositions and methods described herein may be used to protect healthy neurological tissue from radiation toxicity due to radiation exposure.

The methods described herein in which the compositions are used as neuroprotective agents (prophylactics) includes, but is not limited to: (i) early diagnosis of patients at risk of developing neurodegenerative diseases and disorders prior to onset of such diseases and disorders, wherein such patients are given a subtherapeutic dose of a composition comprising a compound (or compounds) of Formula (I) on a periodic basis to protect healthy neural tissue from developing such diseases and disorders; and (ii) after onset of a neurodegenerative diseases and disorders the patient initially receives a therapeutic dose of a composition comprising a compound (or compounds) of Formula (I) to stop the progression of such diseases and disorders, and wherein such patients are then given a subtherapeutic dose of a composition comprising a compound (or compounds) of Formula (I) on a periodic basis to protect healthy neural tissue from further progression.

In certain embodiments the periodic administration of a subtherapeutic dose of compounds of Formula (I), compositions comprising a compound (or compounds) of Formula (I), or drug combinations described herein that include compounds of Formula (I) includes, but is not limited to administration daily, every two days, every three days, every four days, every five days, every six days, once a week, twice a month, and once a month.

In certain embodiments the administration of a therapeutic dose of compounds of Formula (I), compositions comprising a compound (or compounds) of Formula (I), or drug combinations described herein that include compounds of Formula (I) includes, but is not limited to administration daily, every two days, every three days, every four days, every five days, every six days, once a week, twice a month, and once a month.

In certain embodiments, the administration of a therapeutic dose of compounds of Formula (I) is temporarily suspended or the drug dose decreased (a “drug holiday”). At the end of the drug holiday, the previous dosing regimen can be restored or further modified. A drug holiday can last, for example, 2 days, 3 days, 4 days, 5 days, 6 days, 7 days, 10 days, 15 days, 20 days, 25 days, 30 days, 40 days, 50 days, or 60 days.

Methods used for the early diagnosis of patients at risk of developing neurodegenerative diseases and disorders include, but are not limited to, genotype analysis, phenotype analysis (using biomarkers or functional markers) or any combination of genotype and phenotype analysis. Genotype analysis can be accomplished by methods known in the art for detecting sequences at polymorphic sites, and therefore patients at risk of developing neurodegenerative diseases and disorders may be selected using genetic markers. The presence or absence of a genetic marker for neurodegenerative diseases and disorders may be determined by various methods, including, for example, using enzymatic amplification, restriction fragment length polymorphism analysis, nucleic acid sequencing, electrophoretic analysis of nucleic acid from the individual, or any combination thereof. In certain embodiments, determination of such genetic markers may identify patients who will respond to, or gain benefit from, treatment with compounds of Formula (I), or drug combinations described herein that include compounds of Formula (I). By way of example, methods of diagnosing a susceptibility to amyotrophic lateral sclerosis in an individual, comprises determining the presence or absence of certain genetic markers, wherein the presence of the genetic marker is diagnostic of susceptibility to develop amyotrophic lateral sclerosis.

Early diagnosis of a patient based on biomarker phenotypes may be used as an alternative to, or as a compliment with, patient screening by genetic marker detection. The term “biomarker” as used herein refers to a characteristic which can be measured and evaluated as an indicator of normal biological processes, pathological processes, or pharmacological responses to therapeutic intervention. Thus a biomarker may be any substance, structure or process which can be measured in the body, or its products, and which may influence or predict the incidence of outcome or disease. Biomarkers may be classified into markers of exposure, effect, and susceptibility. Biomarkers can be physiologic endpoints or they can be analytical endpoints. Techniques, used to monitor and/or measure biomarkers include, but are not limited to, NMR, LC-MS, LC-MS/MS, GC-MS, GC-MS/MS, HPLC-MS, HPLC-MS/MS, FT-MS, FT-MS/MS, ICP-MS, ICP-MS/MS, peptide/protein sequencing, nucleic acid sequencing, electrophoresis techniques, immuno-assays, immuno-blotting, in-situ hybridization, fluorescence in-situ hybridization, PCR, radio-immuno assays, and enzyme-immuno assays. Single nucleotide polymorphisms (SNPs) may also been useful for the identification of biomarkers for propensity to certain neurodegenerative diseases or disorders. These techniques, or any combination thereof, may be used to early diagnose patients for risk of developing neurodegenerative diseases and disorders, wherein such patients may be beneficially treated with compounds of Formula (I), or drug combinations described herein that include compounds of Formula (I).

Early diagnosis of a patient based on the evaluation of functional markers may be used as an alternative to, or as a compliment with, patient screening by genetic marker detection (genotype analysis) and/or monitoring/measurement of biomarker phenotypes. Functional markers may include, but are not limited to, any physical characteristics associated with a neurodegenerative disease or disorder. By way of example only, the slurring of speech may be used as a functional marker for amyotrophic lateral sclerosis. These techniques, or any combination thereof, may be used to early diagnose patients for risk of developing neurodegenerative diseases and disorders, wherein such patients may be beneficially treated with compounds of Formula (I), or drug combinations described herein that include compounds of Formula (I).

Disclosed herein are methods and compositions used as neuroprotective agent and/or to treat amyotrophic lateral sclerosis comprising administration of a therapeutic dose of compounds of Formula (I), compositions comprising a compound (or compounds) of Formula (I), or drug combinations described herein that include compounds of Formula (I).

Disclosed herein are methods and compositions used as neuroprotectiye agent and/or to treat dementia and or AIDS dementia comprising administration of a therapeutic dose of compounds of Formula (I), compositions comprising a compound (or compounds) of Formula (I), or drug combinations described herein that include compounds of Formula (I).

Disclosed herein are methods and compositions used as neuroprotective agent and/or to treat Parkinson's disease comprising administration of a therapeutic dose of compounds of Formula (I), compositions comprising a compound (or compounds) of Formula (I), or drug combinations described herein that include compounds of Formula (I).

Disclosed herein are methods and compositions used as neuroprotective agent and/or to treat motor neuron disorders comprising administration of a therapeutic dose of compounds of Formula (I), compositions comprising a compound (or compounds) of Formula (I), or drug combinations described herein that include compounds of Formula (I).

Disclosed herein are methods and compositions used as neuroprotective agent and/or to treat Huntington's disease comprising administration of a therapeutic dose of compounds of Formula (I), compositions comprising a compound (or compounds) of Formula (I), or drug combinations described herein that include compounds of Formula (I).

Formulations, Routes of Administration, and Effective Doses

For convenience, the compositions, formulations, routes of administration, and effective doses described in this section have been described generically and/or with specific examples. However the compositions, formulations, routes of administration, and effective doses described in this section should not be limited to just the generic descriptions or specific example provided in this section, but rather the compositions, formulations, routes of administration, and effective doses described in this section apply equally well to all compounds that fall within the scope of Formulas I-V, including any sub-formulas or specific compounds that fall within the scope of Formulas I-V that are described in the specification, claims and figures herein.

The compounds of Formula (I) may be administered in the form of pharmaceutical compositions, wherein such pharmaceutical compositions may include at least one of the following components: one or more of the compounds of Formula (I) as the active ingredient; a pharmaceutically acceptable salt and/or coordination complex thereof; and one or more pharmaceutically acceptable excipients, carriers, which include but ar not limited to inert solid diluents and fillers, diluents, including sterile aqueous solution and various organic solvents, permeation enhancers, solubilizers and adjuvants. Such compositions may be prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.). In addition, the compounds of Formula (I) may be administered alone or in combination with other therapeutic agents. Such pharmaceutical compositions and combination therapies can be used to treat neurodegenerative diseases and disorders in the methods as described herein.

The compounds of Formula (I) may be provided as a prodrug and which may interconvert to compound of Formula (I) in vivo after administration. The compounds of Formula (I) and/or its prodrug, or its pharmaceutically acceptable salts may be used in developing a formulation for use in the methods disclosed herein. Further, the compounds of Formula (I) may undergo intracellularly ligand exchange with a ligand derived from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. Further, in some embodiments, the compound may be used in combination with one or more other compounds or in one or more other forms. The compound of Formula (I) may be formulated, in the same dosage unit e.g. in one cream, intravenously-suitable formulation, solution, suppository, tablet, a lyophilized powder suitable for reconstitution into a solution, or capsule.

The compositions of Formula (I) may be administered in either single or multiple doses by any of the accepted modes of administration of agents having similar utilities, for example as described in those patents and patent applications incorporated by reference above, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, including, by way of example, by injection. The forms in which the pharmaceutical compositions of Formula (I) may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

In a further or alternative embodiment of the parenteral formulation of Formula (I), the solution further comprises an acid. In another embodiment of the parenteral formulation of Formula (I), the acid is acetic acid. In yet another embodiment of the parenteral formulation of Formula (I), the acid is acetic acid and the solution has a pH between about 4.5 and about 5.5; or between about 4.7 and 5.3. In still yet another embodiment of the parenteral formulation of Formula (I), the acid is acetic acid and the solution has a pH between about 4.5 and about 5.5.

In a further or alternative embodiment of the parenteral formulation of Formula (I), the solution further comprises an isotonic agent. In a further or alternative embodiment of the parenteral formulation of Formula (I), the isotonic agent is selected from the group consisting of saccharides, polyhydric alcohols, and dibasic sodium phosphate. In a further or alternative embodiment of the parenteral formulation of Formula (I), the isotonic agent is a polyhydric alcohol selected from the group consisting of mannitol and sorbitol. In a further or alternative embodiment of the parenteral formulation of Formula (I), the isotonic agent is about 3-10% mannitol; in another embodiment, about 4-6% mannitol.

In a further or alternative embodiment of the parenteral formulation of Formula (I), the concentration of the compound of Formula (I) is between about 2.0 mg/nL and about 3.0 mg/mL; between about 2.2 mg/mL and about 2.8 mg/mL; between about 2.3 mg/mL and about 2.7 mg/mL; or between about 2.4 mg/mL and about 2.6 mg/mL. In a further or alternative embodiment of the parenteral formulation of Formula (I), the concentration of the compound of Formula (I) is about 2.5 mg/mL. In a further or alternative embodiment, the concentration of the compound of Formula (I) is about 2.0 mM, or about 2.2 mM, or about 2.4 mM.

At high concentrations, texaphyrins have a tendency to aggregate in aqueous solution, which potentially decreases their solubility. Aggregation (self-association) of polypyrrolic macrocyclic compounds, including porphyrins, sapphyrins, texaphyrins, and the like, is a common phenomenon in water solution as the result of strong intermolecular van der Waals attractions between these flat aromatic systems. Aggregation may significantly alter the characteristics of the macrocycles in solution. Addition of a carbohydrate, saccharide, polysaccharide, or polyuronide to the formulation decreases the tendency of the texaphyrin to aggregate, thus increasing the solubility of the texaphyrin in aqueous media. Examples of such agents are sugars, including mannitol, dextrose or glucose. In one embodiment, mannitol is used at concentrations of about 2-8% concentration. In certain embodiments mannitol is used at concentrations of about 5%. Such aqueous solutions are suitable for intravenous, intramuscular, subcutaneous and intraperitoneal administration.

Prolonged absorption of the injectable compositions can be brought about by the use in the compositions of agents delaying absorption, for example, aluminum monostearate and gelatin. These particular aqueous solutions are suitable for intra-arterial, intravenous, intramuscular, subcutaneous and intraperitoneal administration.

Sterile injectable solutions are prepared by incorporating the compositions of Formula (I) in the required amount in the appropriate solvent with various other ingredients as enumerated above, as required, followed by sterile filtration. Generally, dispersions are prepared by incorporating sterilized compositions of Formula (I) into a sterile vehicle which contains the dispersion medium and the required other ingredients from those enumerated herein. In the case of sterile powders for the preparation of sterile injectable solutions, the methods of preparation are vacuum-drying and freeze-drying techniques, which yield a powder of the compositions of Formula (I) plus any additional desired ingredient from a previously sterile-filtered solution thereof.

The compositions of Formula (I) may be impregnated into a stent by diffusion, for example, or coated onto the stent such as in a gel form, for example, using procedures known to one of skill in the art in light of the present disclosure.

Oral administration is another route for administration of the compositions of Formula (I). Embodiments include oral administration via capsule or enteric-coated tablets, or the like, which prevent degradation of the compositions of Formula (I) in the stomach. In making the pharmaceutical compositions that include at least one composition of Formula (I), the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the compositions of Formula (I). Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the compositions of Formula (I), soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of Formula (I) can be formulated so as to provide quick, sustained or delayed release of the compositions of Formula (I) after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods described herein employs transdermal delivery devices (“patches”). Such transdermal patches may be used to provide continuous or discontinuous infusion of the compositions of Formula (I) in controlled amounts, see, e.g., U.S. Pat. Nos. 5,023,252, 4,992,445 and 5,001,139. Such patches may be constructed for continuous, pulsatile, or on demand delivery of pharmaceutical agents.

The compositions may be optionally formulated in a unit dosage form. The term “unit dosage form(s)” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, a capsule, and/or an ampoule). The compositions of Formula (I) are effective over a wide dosage range and are generally administered in a pharmaceutically effective amount. The specific dose will vary depending on the particular compound of Formula (I) chosen, the dosing regimen to be followed, and the apical ligands chosen, because of the wide range of properties available, such as solubilities, lipophilicity properties, lower toxicity, and improved stability. By way of example only, dosages within the range of about 0.01 mg/kg/treatment up to about 100 mg/kg/treatment may be used, and in certain embodiments about 0.1 mg/kg/treatment to about 50 mg/kg/treatment may be used. In addition, in certain embodiments of oral administration, each dosage unit may contain from about 10 mg to about 2 g of a composition of Formula (I), while in certain embodiments for parenteral administration, each dosage unit may contain from about 10 mg to about 700 mg of a composition of Formula (I). In certain parenteral administration embodiments the dosage unit is about 350 mg. The amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the composition of Formula (I) is mixed with a pharmaceutical excipient to form a solid preformulation composition containing a homogeneous mixture of a composition of Formula (I). When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. Such compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, orally or nasally, from devices that deliver the formulation in an appropriate manner.

For convenience, the packaging, packaged product and the form of the packaged product described in this section have been described generically and/or with specific examples. However the packaging, packaged product and the form of the packaged product described in this section should not be limited to just the generic descriptions or specific example provided in this section, but rather the packaging, packaged product and the form of the packaged product described in this section apply equally well to all compounds that fall within the scope of Formulas I-V, including any sub-formulas or specific compounds that fall within the scope of Formulas I-V that are described in the specification, claims and figures herein.

The types of outer packaging, types of containers, qualification of standards for containers, forms of packaged compounds of Formula (I), and general packaging specifications used in the packaging of compounds of Formula (I) or compositions comprising a compound (or compounds) of Formula (I) are described in U.S. patent application Ser. No. 11/241,549, which is herein incorporated by reference in its entirety.

Compounds of Formula (I) or compositions comprising a compound (or compounds) of Formula (I) for use as neuroprotective agents (prophylactics) and/or to treat such neurodegenerative diseases and disorders may be prepared for packaging in different forms, including by way of example only, as a solution or a powder. Depending on the form of Formula (I), an appropriate container suitable to hold compounds of Formula (I) or compositions comprising a compound (or compounds) of Formula (I) may be used. Also dependent upon the container chosen, sealing the container and adjusting the environment inside the container for packing will be done. Optional steps may involve adding extra materials either to the container or along with the container for packaging, by way of example only includes a bottle top, desiccants, tamper-proof seal, plastic wrap and the like. Finally the sealed container containing compounds of Formula (I) or compositions comprising a compound (or compounds) of Formula (I) is packaged within an appropriate outer package.

In one embodiment, the vial or container that contains the compound of Formula (I) has a seal and fits into an outer packaging. The container aids to protect its contents of Formula (I) from contaminants, degradation, impurities, other solutions and/or spillage. Further or alternative embodiments of different container types include, by way of example only, a high density polyethylene container, a plastic bottle, a syringe, a “drip bag,” a pre-filled syringe, an intravenous bag, and the like. In one embodiment, the outer packaging is a paper box, while in another embodiment, the outer packaging protects the container with seal and contents (a solution of Formula (I)) from light. In further or alternative embodiments, the outer packaging protects the container with a seal along with an aluminum seal protector and its contents of Formula (I) from sunlight, ultraviolet light, contaminants, degradation, impurities, other solutions and spillage. The outer packaging will not significantly absorb, react with, or otherwise adversely affect the Formula (I) drug or other excipients or components used in intravenous delivery during storage of the drug prior to its use. The outer packaging may be in any shape or form which protects container with seal and its contents of Formula (I), including, by way of example only a paper box, a cardboard box, a carton, a plastic bag, a fabric case, a metal receptacle, a wooden bin or the like.

Further or alternative embodiments, by way of example only, include a combination of a syringe sealed in plastic with a cardboard box, a combination of a syringe sealed in plastic with an outer nontransparent paper lining, a combination of a glass bottle sealed in plastic with a cardboard box, a combination of a plastic bottle with a cardboard box, a combination of a plastic bottle sealed in plastic with a cardboard box, a combination of a glass bottle encased in a Styrofoam case within a cardboard box, a combination of a syringe encased in a Styrofoam case within a cardboard box, and the like. The qualification standards for other such combinations of sealed containers and outer packaging differ because of the different materials used in the container and outer packaging. However, any combination should provide protection from contamination, such as the crystallization or degradation, of the drug, and from other environmental factors, during storage of the system prior to its use. Further, the outer packaging may contain a desiccant or an oxygen-absorbing material.

The qualification standards for a vial or sealed container varies depending on the type of vial or sealed container used and which form of Formula (I) is used. By way of example only, a sealed syringe housing a powder form of Formula (I) or a sealed bottle housing a powder form of Formula (I) may withstand higher temperatures than a sealed syringe housing a liquid form of Formula (I) or a sealed bottle housing a liquid form of Formula (I) which may lead to a higher rate of degradation of the drug. In one embodiment, the container housing the drug is in an oxygen depleted environment which is sealed and substantially airtight. However, any combination should provide protection from contamination, such as the crystallization or degradation, of the drug, and from other environmental factors, during storage of the system prior to its use.

In one embodiment, the liquid form of Formula (I) is housed in a container with a minimal amount of headspace for storage. The headspace may contain at least about 90% nitrogen gas, or at least about 95% nitrogen gas and occupy either less than about 12% or less than about 7% of the volume of the sealed container. In still a further embodiment, the liquid form of Formula (I) is flushed with nitrogen inside the container. In a further embodiment, a non-oxygen gas (including nitrogen, argon, neon or combinations thereof) is flushed into the empty container followed by the solution of Formula (I); alternatively, the solution of Formula (I) partially fills the container and the remaining head space is flushed with a non-oxygen gas.

In further or alternative embodiments, a protective cap may accompany the bottle seal or syringe tip seal. The protective cap may prevent unintentional damage to the bottle or syringe tip seal before use. In another embodiment, the protective cap may be child-resistant to prevent unintentional opening by a minor before use. In still further or alternative embodiments, a plastic bag, a foil wrapped container or other such materials may seal the vial and/or sealed container within the outer packaging. The plastic bag or foil wrapped container may provide another protective layer against light, contaminants, degradation, impurities, other solutions and spillage.

Any of the pharmaceutical compositions and formulations described herein may be packaged as described herein. One embodiment described herein is a packaged product of Formula (I) for intravenous drug use to a human subject wherein the packaging will not significantly absorb, react with, or otherwise adversely affect the drug or other excipients or components used in intravenous delivery during storage of the system prior to its use. The foregoing and other objectives are achieved by providing light protective materials and a substantially deoxygenated environment to prevent degradation to Formula (I) prior to use. Such light protective materials include an outer packaging that is opaque and an inner package that comprises a transparent, non-tinted material, such as glass.

In further or alternative embodiments, storage-stabilized formulation contains an isotonic agent, which can include electrolytes and/or non-electrolytes. Non-limiting examples of electrolytes includes sodium chloride, potassium chloride, dibasic sodium phosphate, sodium gluconate and combinations thereof. Non-limiting examples of non-electrolytes includes saccharides and polyhydric alcohols; further examples include mannitol, sorbitol, glucose, dextrose, glycerol, xylitol, fructose, maltose, mannose, glycerin, propylene glycol, and combinations thereof. In still further embodiments, the storage-stabilized formulation comprises a buffer, an anti-crystallizing agent, and/or a preservative. Buffering agents aid in stabilizing pH. Anti-crystallizing agents aid in stabilizing the concentration of the solution. Preservatives aid in preventing the growth of micro-organisms, and include by way of example only, methyl paraben, propyl paraben, benzyl alcohol, sodium hypochlorite, phenoxy ethanol and/or propylene glycol. In one, the storage-stabilized formulation does not contain an oxidizing agent other than Formula (I) and oxygen. Oxidizing agents promote degradation of the compound of Formula (I).

The packaging system may be prepared by loading the product package contents (i.e., Formula (I), bottle, syringe, plastic bag, desiccant, cardboard box) by means of any suitable or conventional manufacturing operation and sealing process. The sealing process may include gas flushing or evacuation of oxygen from the container.

In one embodiment, Formula (I) may be packaged in powder form with reconstituting solution. Reconstitution is achieved by admixing the Formula (I) powder with a solution comprising, by way of example, water, acetic acid and mannitol, using amounts and concentrations as described for the Formula (I) solutions described herein. The term “powder” is used to generically describe any solid form of Formula (I) in a particulate form, including crystalline forms and non-crystalline forms, or grains, beads, chunks, fine powders, coarse powder or other particulate forms.

In one embodiment, the container is a non-tinted borosilicate glass vial, USP Type I. The vial can hold a sufficient amount of a solution of Formula (I) to allow reliable administration of 50 mL of such a solution to a patient (which generally means the vial can hold 51-53 mL of solution). Further, such a vial has a suitable head space and an opening of 20 mL Further, the seal for the container is a one piece elastomeric bottle stopper composed of butyl rubber which forms a tight seal onto a glass bottle container housing Formula (I). In this embodiment, the stopper is a 20 mm flange type constructed from 4405/50 gray butyl rubber and laminated at the product contact area with a Teflon® film. Teflon® is fluorinated ethylene-propylene (FEP) applied as a film to the face of the stopper. The seal diameter is 20 mm and the seal is constructed of aluminum with a violet colored plastic Flip-Off® button.

Administration for Photodynamic Therapy

By way of example, a composition of Formula (I), wherein the metal used allows for photodynamic therapy, may be administered in solution, optionally in 5% mannitol USP. Such metals include, but are not limited to, lutetium as the metal in the texaphyrin. Dosages of about 1.0 to 2.0 mg/kg to about 4.0 to 7.0 mg/kg, including 3.0 mg/kg, are employed, although in some cases a maximum tolerated dose may be higher, for example about 5 mg/kg. The texaphyrin is administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

Dose levels for certain uses may range from about 0.05 mg/kg to about 20 mg/kg administered in single or multiple doses (e.g., before each fraction of photoirradiation). The lower dosage range would be applicable, for example, to intra-arterial injection or for impregnated stents.

The optimum length of time between administration of such compositions of Formula (I) and light treatment can vary depending on the mode of administration, the form of administration, and the type of target tissue. Typically, the compositions of Formula (I) persists for a period of minutes to hours, depending on the composition of Formula (I), the formulation, the dose, the infusion rate, as well as the type of tissue and tissue size. The light source for the photodynamic therapy may be a laser, a light-emitting diode, or filtered light from, for example, a xenon lamp; and the light may be administered topically, endoscopically, or interstitially (via, e.g., a fiber optic probe), or intraarterially.

The co-administration of an anti-emetic, a sedative (e.g., benzodiazapenes) and narcotics/analgesics are sometimes recommended prior to light treatment along with topical administration of a local anesthetic, for example Emla cream (lidocaine, 2.5% and prilocalne, 2.5%) under an occlusive dressing. Other intradermal, subcutaneous and topical anesthetics may also be employed as necessary to reduce discomfort. Subsequent treatments can be provided after approximately 21 days.

When employing photodynamic therapy, a target area is treated with light, for example at about 740±16.5 nm. After the photosensitizing composition of Formula (I) has been administered, the tissue being treated is photo irradiated at a wavelength similar to the absorbance of the composition of Formula (I), usually either about 440 to 540 nm or about 700 to 800 nm, or about 450 to 520 nm, or about 720 to 780 nm, or about 460 to 500 nm or about 725 to 760 nm. The light source may be a laser, a light-emitting diode, or filtered light from, for example, a xenon lamp; and the light may be administered topically, endoscopically, or interstitially (via, e.g., a fiber optic probe), or intra-arterially. In one embodiment, the light is administered using a slit-lamp delivery system. The fluence and irradiance during the photo irradiating treatment can vary depending on the type of tissue, depth of target tissue, and the amount of overlying fluid or blood. For example, a total light energy of about 100 J/cm2 can be delivered at a power of 200 mW to 250 mW, depending upon the target tissue.

Administration for Radiation Sensitization

Compositions of Formula (I), wherein the metal used allows for radiation sensitization, may be administered in a solution containing about 2 to 2.5 mM of the compound of Formula (I), optionally in 5% mannitol USP/water (sterile and non-pyrogenic solution); in a further or alternative composition, the solution contains about 2.2-2.6 mM of the compound of Formula (I) (in addition to other components described herein). Such metals include, but are not limited to, the metal is gadolinium as the metal in the texaphyrin. Dosages of 0.1 mg/kg up to as high as about 29.0 mg/kg have been delivered, and in certain embodiments about 3.0 to about 15.0 mg/kg (for volume of about 90 to 450 mL) may be employed, optionally with pre-medication using anti-emetics when dosing above about 6.0 mg/kg. The compound may be administered via intravenous infusion over about a 5 to 10 minute period, followed by a waiting period of about 2 to 5 hours to facilitate intracellular uptake and clearance from the plasma and extracellular matrix prior to the administration of radiation.

When employing whole brain radiation therapy, a course of 30 Gy in ten (10) fractions of radiation may be administered over consecutive days excluding weekends and holidays. In the treatment of brain metastases, whole brain megavolt radiation therapy is delivered with 60 Co teletherapy or a ≦4 MV linear accelerator with isocenter distances of at least 80 cm, using isocentric techniques, opposed lateral fields and exclusion of the eyes. A minimum dose rate at the midplane in the brain on the central axis is about 0.5 Gy per minute.

Compositions of Formula (I) used as radiation sensitizers may be administered before, or at the same time as, or after administration of the ionizing radiation. The composition of Formula (I) may be administered as a single dose, as an infusion, or it may be administered as two or more doses separated by an interval of time. Where the composition of Formula (I) is administered as two or more doses, the time interval between the composition of Formula (I) administrations may be from about one minute to a number of days, from about 5 minutes to about 1 day, or from about 10 minutes to about 10 hours. The dosing protocol may be repeated, from one to ten or more times, for example. Dose levels for radiation sensitization may range from about 0.05 mg/kg to about 20 mg/kg administered in single or multiple doses (e.g. before each fraction of radiation). The lower dosage range would be preferred for intra-arterial injection or for impregnated stents.

Combination Therapies

For convenience, the combination therapies described in this section have been described generically and/or with specific examples. However the combination therapies described in this section should not be limited to just the generic descriptions or specific example provided in this section, but rather the combination therapies described in this section apply equally well to all compounds that fall within the scope of Formulas I-V, including any sub-formulas or specific compounds that fall within the scope of Formulas I-V that are described in the specification, claims and figures herein.

Compounds of Formula (I) may be administered to a patient in conjunction with anti-inflammatory agents, including by way of example only, indomethacin, acetylsalicylic acid (aspirin), ibuprofen, sulindac, phenylbutazone, naproxen, diclofenac, celecoxib, resveratrol, CAY 10404 and curcumin. When administered in a combination, the compound of Formula (I) can be administered before, simultaneously and/or after the anti-inflammatory agent. The time between administration of a compound of Formula (I) and administration of an anti-inflammatory agent can be between 0 seconds (i.e., the two agents are administered simultaneously) to 1 week. When administered simultaneously, the two agents may be given in the same pharmaceutical dose or in separate pharmaceuticals doses.

Zinc is a co-factor in a variety of cellular processes including DNA synthesis, behavioral responses, reproduction, bone formation, growth and wound healing. Zinc is a component of insulin and it plays a role in the efficacy of most of the functions of your body. Zinc is necessary for the free-radical quenching activity of superoxide dismutase (SOD), an antioxidant enzyme which breaks down the free-radical superoxide to form hydrogen peroxide. The abundance of loosely-bound or free intracellular zinc can impact on cellular metabolism, survival and growth. Zinc might aid in the prevention and treatment of cancer. The methods described herein provide for a method of treating neurological diseases and disorders and/or free-radical associated diseases and disorders, which involves the administration of a combination of an effective amount of metal containing texaphyrin of Formula (I) and an effective amount of a zinc compound. Examples of zinc compounds that can be used in the methods of the present invention include, but are not limited, to zinc acetate, zinc chloride, zinc citrate, zinc lactate zinc gluconate, L-camosine salt, zinc fetuin, zinc sulfate, zinc bacitracin, zinc seleno-bacitracin, chelated zinc, zinc complex of 1-hydroxypyridine-2-thione, and zinc ionophores such as zinc 1-hydroxypyridine-2-thiol. For further details on the administration of compounds of Formula (I) in conjunction with zinc reagents see U.S. Patent Application No. 60/737,601 and International Application No. PCT/US/2005/017812, each of which is incorporated by reference in its entirety. When administered in a combination, the compound of Formula (I) can be administered before, simultaneously and/or after the zinc reagent. The time between administration of a compound of Formula (I) and administration of a zinc reagent can be between 0 seconds (i.e., the two agents are administered simultaneously) to 1 week. When administered simultaneously, the two agents may be given in the same pharmaceutical dose or in separate pharmaceuticals doses.

The zinc compound is effective over a wide dosage range and is generally administered in a pharmaceutically effective amount. By way of example only, for oral administration, each dosage unit contains from 40-100 μmol/kg of the zinc compound, It will be understood, however, that the amount of the metal containing texaphyrin and/or zinc compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

A compound of Formula (I) may also be administered to a patient in conjunction with other neurological therapeutic agents used as neuroprotective agents or to treat neurodegenerative diseases and disorders. Such therapeutic agents include, but are not limited to, antioxidants, glutamate antagonists, metal chelators, neural growth factors, non-neural growth factors, calcium regulators, anti-inflammatory agents, inhibitors of cell signalling pathways, inhibitors of cell death pathways, dietary supplements, energetic precursors (by way of example creatine), immunoregulatory agents, chloinergic agents, dopaminergic agents and anti-viral agents. Compound of Formula (I) may be administered before, at the same time as, or after administration of one or more neurological therapeutic agents. The compound of Formula (I) may be administered as a single dose, or it may be administered as two or more doses separated by an interval of time. The compound of Formula (I) may be administered concurrently with, or from about 1 minute to about 12 hours following administration of a neurological therapeutic agent, preferably from about 5 minutes to about 5 hours, more preferably about 4 to 5 hours. The dosing protocol may be repeated, from one to three times, for example. A time frame that has been successful in vivo is administration of a compound of Formula (I) 1 about 5 minutes and about 5 hours after administration of a neurological therapeutic agent, with the protocol being performed once per week for three weeks. Administration may be intra-arterial injection, intravenous, intraperitoneal, intramuscular, subcutaneous, oral, topical, or via a device such as a stent.

The choice of therapy that can be co-administered with the compositions disclosed herein will depend, in part, on the disease or disorder being treated.

The compounds, formulations and methods described herein are useful in the treatment of conditions and diseases associated with free-radical species, by way of example due to elevated concentrations of reactive oxygen species such as OH (hydroxyl radicals), H₂O₂ (peroxide), ° 2—(super oxide radical anion), NO— (nitric oxide), or ⁻OONO (peroxynitrite), including but not limited to:
treating neurological diseases, such as Alzheimers, Parkinson's, amyotrophic lateral sclerosis (ALS) and multiple sclerosis (MS);
treating inflammatory diseases of immune and autoimmune origins, e.g., involving excessive phagocytosis, such as rheumatoid arthritis, graft-vs-host disease.
treating tissues experiencing a physical or chemical insult, including the treatment of cardioplegia, hypoxic and/or reperfusion injury to cardiac or skeletal muscle or brain tissue (e.g., stroke), and use in and after transplants;
treating shock conditions (including cardiogenic shock);
protecting skeletal muscle against damage, e.g., resulting from trauma, or damage subsequent to muscle or systemic diseases;
protecting myocardial tissue against ischaemic damage in subjects with myocardial infarction, especially in patients who are waiting to receive treatments such as thrombolytic drugs or PTCA (percutaneous transluminal coronary angioplasty);
protecting neuronal tissue against ischaemia resulting from cardiac function impairment or from non-cardiac conditions (including protecting brain tissue against ischaemia-induced metabolic disorders); and
preserving donor tissues for use in transplants (protecting them from the deleterious effects of ischaemia), by administration to the donor, the recipient and/or by adding to the ex-vivo perfusion fluid an effective amount of a compound of Formula (I), or a pharmaceutically acceptable salt thereof, particularly for renal transplants, skin grafts, cardiac transplants, lung transplants, corneal transplants, and liver transplants.

Use of the compounds, formulations and methods of the present invention can also entail the co-administration of a compound of Formula (I) together with another pharmaceutically active agent, such as a thrombolytic agent [especially TPA (Tissue Plasminogen Activator) or streptokinase] or an anti-anginal (such as beta blockers, including propranolol and timolol).

In certain embodiments the compounds of Formula (I) may be used to in diagnostic evaluation of neurodegenerative diseases and disorders and/or free-radical associated diseases and disorders. Such diagnostic evaluation includes, but not limited to, imaging methods and techniques such as, by way of example only, magnetic resonance imaging (MRI). Compounds of Formula (I) may be administered to a patient up to 1 hour, up to 2 hours, up to 4 hours, up to 6 hours, up to 8 hours, up to 10 hours, up to 12 hours, within 24 hours, 1 day, 2 days, 3 days, 5 days, or up to 10 days, prior to diagnostic evaluation. Compounds of Formula (I) may be administered to a patient up to 1 hour, up to 2 hours, up to 4 hours, up to 6 hours, up to 8 hours, up to 10 hours, up to 12 hours, within 24 hours, 1 day, 2 days, 3 days, 5 days, or up to 10 days, prior to magnetic resonance imaging.

Testing

Activity testing is conducted as described below, in the cited references, and by modifications thereof.

Ischaemia Assays

Cerebral ischaemia is the result of either a generalized or a localized prolonged reduction of blood flow to the brain. Such a blood flow reduction can result from various pathological conditions including cerebral venous inflammation and thrombosis, cardiac diseases, changes in blood (clotting, viscosity, anaemia) or from cardiac surgical practices. One of the indications of damage produced by cerebral ischaemia is the increase of the iso-enzyme creatinephosphokinase 1 (CPK₁) in the plasma (Rossi, et al., Am. J. Cardiol., 58(13):1236-1241 (1986)). Inhibition of the peripheral appearance of CPK₁ is an indication of reduced damage caused by ischaemia to the brain. This is demonstrated by administration of a test compound prior to coronary artery ligation in the baboon, as a bolus i.v. injection followed by an infusion over the period of reperfusion, as described by Alps, et al., (Arzneim. Forsch Drug Res., 33(1)6: 868-876 (1983)).

In vivo protective effects against the deleterious effects of cerebral ischemia are determined by use of the standard gerbil brain ischemia model or modifications thereof. (See, T. Kirino, Brain Res. 239:57-69P (1982)). Another in vivo assay is the Middle Cerebral Artery Occlusion Model as described, for example, in WO 00/09512.

Protection of the myocardium against ischaemic damage is experimentally demonstrated by inducing infarction in a suitable test animal (e.g., a baboon) followed by examination of insult-induced elevations in enzyme levels (particularly creatine kinase “CK” and lactate dehydrogenase “LDH”). It is accepted that concentrations of these enzymes are increased after myocardial damage (Galen, et al., J.A.M.A., 232:145-147 (1975)) and that such enzyme levels can be measured by an experimental test that is an adaptation of the one described by Alps, et al. (Arzneim. Forsch Drug Res., 33, (1), 6, 868-876, 1983)).

Protection against myocardial ischaemia can also be assessed via effectiveness to prevent ischaemia-induced increase in alpha-1 adrenoceptor number in the myocardium. It is known that alpha-1 adrenoceptor population increases in the myocardium suffering from ischaemia (Heathers, et al., Circulation Research, 61, 735-746 (1987)). It has also been shown that alpha-1 adrenoceptor antagonists have beneficial effects during ischaemia in animal models (Wilbur, et al., J. Cardiovascular Pharmacol., 10, 96-106, (1987)). Thus agents which prevent the ischaemia-induced increase in alpha-1 adrenoceptors density are beneficial during myocardial ischaemia. The ability of compounds to inhibit the ischaemia-induced increase of alpha-1 adrenoceptors in myocardium can be assessed in the rat left ventricle using a model of ischaemia described by Allely and Brown (Br. J. Pharmacol., 95:705P (1988)).

Traumatic Ischaemia Assays

Protection of the myocardium against deleterious effects of ischaemia induced by open-heart and other cardiac surgical procedures, including cardioplegia, can be assessed by a method modified from Langendorff, which entails measuring coronary effluent pH and lactate level. These tracers are recognized as indicative of tissue damage induced by severe reduction in the nutrient supply to the heart (Armiger, et al., Biochem. Med., 29:265-267 (1983); van Gilst, et al., Archives of Pharmacol., suppl., 330:161P (1985)).

Protection of skeletal muscles against damage resulting, for example, from major surgical practices, can be experimentally assessed in the same model used to assess protective effects at the myocardial level. For this purpose skeletal muscle-specific isoenzymes CK₃ and LDH₅, are assayed as indications of damaged muscle (Galen, Med. Times, 105(2):89-99 (1977)).

Utility in the preservation of organs for transplantation is demonstrated by administering the test compound to pigs before nephrectomy, and/or by adding the compound to the fluid used for flushing and storage of the organ and by assessing functionality of transplanted kidneys over a period of 14 days. Improvement of renal function in treated animals is assessed by measurement of the glomerular filtration rate and also by peak serum levels for creatinine and urea. Glomerular filtration is a well established indicator of renal function (see, e.g., Mudge and Weiner in The Pharmacological Basis of Therapeutics, Goodman and Gilman, 879, 7th Ed, 1985) and it is generally assessed by measurement of inulin and/or creatinine clearance (Textbook of Medicine, 1088-93, 14th Ed., 1975—Beeson and McDermott Editors).

Anti-Inflammatory, Immunosuippressant and Like Assays

General anti-inflammatory, anti-viral, anti-tumor, anti-psoriatic and/or immunosuppressive activity is associated with the inhibition of Inosine 5′-Monophosphate Dehydrogenase (“IMPDH”). In vitro assays measuring the inhibition of IMPDH, for example, by determining the level of NADH formation according to the method of Anderson, J. H. and Sartorelli, A. C. (J. Biol. Chem., 243:4762-4768 (1968)) are predictive of such activity.

Initial in vivo screening tests to determine anti-inflammatory activity potential include the adjuvant arthritis assay, e.g., according to the method of Pearson (Proc. Soc. Exp. Biol. Med., 91:95-101 (1956)) and the well known carrageenan-induced paw edema assay. Also, in vitro tests, for example those using synovial explants from patients with rheumatoid arthritis (Dayer, et al., J. Exp. Med., 145:1399-1404 (1977)) are useful in determining whether compounds exhibit anti-inflammatory activity.

Immunosuppressive activity is determined by both in vivo and in vitro procedures. In vivo activity is determined, e.g., utilizing a modification of the Jeme hemolytic plaque assay, [Jeme, et al., “The agar plaque technique for recognizing antibody producing cells,” Cell-bound Antibodies, Amos, B. and Kaprowski, H. editors (Wistar Institute Press, Philadelphia) 1963, p. 109]. In vitro activity is determined, e.g., by an adaptation of the procedure described by Greaves, et al. (“Activation of human T and B lymphocytes by polyclonal mitogens,” Nature, 248:698-701 (1974)).

Autoimmune activity is determined, for example, utilizing experimental allergic encephalomyelitis, by a modification of a procedure initially described by Grieg, et. al. (J. Pharmacol. Exp. Ther., 173:85 (1970)).

Activity to prevent the rejection of organ or tissue allografts in experimental animals is determined, for example, as described by Hao, et al. (J. Immunol., 139:4022-4026 (1987)). In addition, U.S. Pat. No. 4,707,443 and EP 226062, incorporated herein by reference, describe assays for activity in prevention of allograft rejection by detection of IL-2R levels. Human clinical trials to establish efficacy in preventing rejection of solid organ transplants (such as renal) are conducted, e.g., as described by Lindholm, Albrechtsen, Tufveson, et al. (“A randomized trial of cyclosporin and prednisolone versus cyclosporin, azathioprine and prednisolone in primary cadaveric renal transplantation,” Transplantation, 54:624-631 (1992)). Human clinical trials for graft vs. host disease are conducted, e.g., as described by Storb, Deeg, Whitehead, et al. (“Methotrexate and cyclosporin compared with cyclosporin alone for prophylaxis of acute graft versus host disease after marrow transplantation for leukemia.” New England J. Med., 314:729-735 (1986)).

While embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### EXAMPLES

### Example 1

### Synthesis of Compound of Formula (I)

All solvents and reagents were of reagent grade quality, purchased commercially, and used as received. ¹³C NMR spectra were recorded using a 250 MHz Varian, 300 MHz GE Tacmag spectrometer, 400 MHz Varian MERCURY or 500 MHz Varian INOVA. UVN is spectra were taken on Beckman DU-640B or Agilent 8453 Spectrophotometer. Column chromatography was run using ICN-Silitech 32-63 D60 Å silica gel or Sorbent Technologies std. activity 50-200μ neutral alumina. Sep-pak reverse-phase tC18 cartridge columns were purchased from Waters.

### Example (1a)

### Preparation of Mn(II) Complex of Formula (I)

Mn(II) texaphyrin complexes are synthesized from the reduced (sp3) texaphyrin precursors using methods, described in WO 95/10307. By way of example 9,10-Diethyl-7,12-dihydro-20,21-bis[2-[2-(2-methoxyethoxy)ethoxy]ethoxy]-4,15-dimethyl-3,6:8,11:13,16-triimino-1,18-benzodiazacycloeicosine-5,14-dipropanol hydrochloride (4,5-Diethyl-10,23-dimethyl-9,24-bis(3-hydroxypropyl)-16,17-bis[2-[2-(2-methoxyethoxy)ethoxy]ethoxy]-13,20,25,26,27-pentaazapentacyclo[20.2.1.1^{3,6}.18,11.0^{14,19}]heptacosa-3,5,8,10,12,14,16,18,20,22,24-undecaene HCl) is stirred in a basic methanol solution with Mn(II) acetate and triethylamine at ambient temperature for several hours (open to the air) to give the (Acetato-Q)[9, 10-diethyl-20,21-bis[2-[2-(2-methoxyethoxy)ethoxy]ethoxy]-4,15-dimethyl-8,11-imino-3,6,16,13-dinitrilo-1,18-benzodiazacyclooeicosine-5,14-dipropanolato-N¹, N¹⁸, N²³, N²⁴, N²⁵]manganese chloride (Mn(II) complex of 4,5-diethyl-10,23-dimethyl-9,24-bis(3-hydroxypropyl)-16,17-bis[2-[2-(2-methoxyethoxy)ethoxy]ethoxy]-13,20,25,26,27-pentaazapentacyclo[20.2.1.1^{3,6}.18,11.0^{14,19}]heptacosa-1,3,5,7,9,11(27),12,14,16,18,20,22(25),23-tridecaene, chloride) counterion which is exchanged for acetate to increase further its aqueous solubility.

Similarly, by substituting the sp3 precursor 4,5-diethyl-9,10,23,24-tetramethyl-16,17-bis(methoxy)-13,20,25,26,27-pentaazapentacyclo[20.2.1.1^{3,6}.18,11.0^{14,19}]heptacosa-3,5,8,10,12,14,16,18,20,22,24-undecaene HCl there is obtained the corresponding Mn(II) complex of 4,5-diethyl-9,10,23,24-tetramethyl-16,17-bis(methoxy)-13,20,25,26,27-pentaazapentacyclo[20.2.1.1^{3,6}.18,11.0^{14,19}]heptacosa-1,3,5,7,9,11(27),12,14,16,18,20, 22(25),23-tridecaene, chloride counterion, which is also exchanged for acetate.

These manganese complexes display a UV-vis spectrum characteristic of aromatic, metallated texaphyrins with a Soret-like band at 460 nm (log ε=4.96 MeOH) and a Q-like band at 727 nm log ε=4.51 MeOH). The positions of these bands are blue-shifted as compared to lanthanide complexes.

### Example (1b)

### Preparation of Co(II) Complex of Formula (I)

In a 500 mL single-neck flask, (9,10-diethyl-7,12-dihydro-20,21-bis[2-[2-(2-methoxyethoxy)ethoxy]ethoxy]-4,15-dimethyl-3,6:8,11:13,16-triimino-1,18-benzodiazacycloeicosine-5,14-dipropanol hydrochloride,) the hydrochloride salt of 4,5,9,24-tetraethyl-16-(7,8-dihydroxyoct-1-yl)oxy-17-methoxy-10,23-dimethyl-13,20,25,26,27-pentaazapentacyclo-[20.2.1.1^{3,6}.1^{8,1}1.0^{14,19}]heptacosa-3,5,8,10,12,14(19),15,17,20,22,24-undecaene) (1.00 g, 1.08 mmol), cobaltous acetate tetrahydrate (323 mg, 1.30 mmol), triethylamine (1.51 mL, 10.8 mmol) and methanol (250 mL) were combined and stirred at ambient temperature open to the atmosphere. After 30 minutes, solvents were removed by rotary evaporation under reduced pressure, and the residue was dried for under high vacuum overnight. The resulting solid was dissolved in 10% acetonitrile in 33 mM ammonium acetate buffer, pH 4.3 (50 mL). This solution of crude complex was loaded onto a Sep-pak™ reverse-phase column (tC 18, 10 g, Waters, Milford, Mass.) prepared with a thin layer cap of Celite™ filter aid. Complex was washed on the column with buffer (100 mL), and deionized water (500 mL), then eluted with methanol (50 mL). Solvent was removed by rotary evaporation under reduced pressure, adding absolute ethanol (50 mL) to azeotrope traces of water, and the residue was dried overnight in vacuo. The resulting green solid was dissolved in methanol (10 mL) and dropped into stirred Et₂O (125 mL) and allowed to stand overnight. The resulting precipitate was collected by filtration and dried in vacuo to provide the cobalt (II) complex of 4,5-diethyl-16,17-bis(2-[2-(2-methoxyethoxy)ethoxy]ethoxy)-9,24-bis(3-acetoxypropyl)-10,23-dimethyl-13,20,25,26,27-pentaazapentacyclo[20.2.1.1^{3,6}.1^{8,11}.0^{14,19}]heptacosa-1,3,5,7,9,11(27),12,14,16,18,20,22(25),23-tridecaene ((acetato-Q)[9, 10-diethyl-20,21-bis[2-[2-(2-methoxyethoxy)ethoxy]ethoxy]-4,15-dimethyl-8,11-imino-3,6,16,13-dinitrilo-1,18-benzodiazacyclooeicosine-5,14-dipropanolato-N¹, N¹⁸, N²³, N²⁴, N²⁵]cobalt) as a green powder (718 mg, 67%). Positive ESI MS (methanol), M⁺: m/e 931 (calc. for C₄₈H₆₆N₅O₁₀Co⁺, 931). Anal.: 59.3; 7.20; 7.23; 4.49 for C, H, N, Co (calc. for C₄₈H₆₆N₅O₁₀Co.C₂H₃O₂.H₂O, 59.52, 7.09, 6.94, 5.84). UV/vis (CH₃OH) [λₘₐₓ, nm (log ε)]: 340 (4.38), 406 shoulder (4.58), 458 (4.83), 672 shoulder (3.97), 716 (4.29). Magnetic moment (Evans): 4.22 B.M.

### Example (1c)

### Preparation of Fe(III) Complex of Formula (I)

In a 500 mL single-neck flask, the hydrochloride salt of 4,5,9,24-tetraethyl-16-(7,8-dihydroxyoct-1-yl)oxy-17-methoxy-10,23-dimethyl-13,20,25,26,27-pentaazapentacyclo-[20.2.1.1^{3,6}.1^{8,11}.0^{14,19}]heptacosa-3,5,8,10,12,14(19),15,17,20,22,24-undecaene (2.00 g, 2.16 nmol), ferrous acetate (452 mg, 2.60 mmol), triethylamine (3.02 mL, 21.6 mmol) and methanol (500 mL) were combined in a round bottom flask fitted with a reflux condenser. The flask was heated at reflux open to the atmosphere for 10 h. UV-visible spectral analysis taken at this time indicated absorbances at 406, 460, and 722 mL After allowing the mixture to cool and stir at ambient temperature overnight, UV-visible spectral analysis indicated absorbances at 408, 456, and 734 nm. Solvents were removed by rotary evaporation under reduced pressure, and the residue was dried under high vacuum overnight. The resulting solid was triturated with acetone (50 mL) for several hours, filtered, and dried overnight. The residue was dissolved in 10% acetonitrile in 33 mM ammonium acetate buffer, pH 4.3 (50 mL). This solution of crude complex was loaded onto two Sep-pak™ reverse-phase columns (tC18, 10 g, Waters, Milford, Mass.) prepared with a thin layer cap of Celite™ filter aid. Complex was eluted from the columns with 30-40% acetonitrile in buffer (200 mL). Organic solvent was partially removed under reduced pressure, and the resulting solution was applied to two fresh reverse-phase columms. The complex was washed on the column with buffer (500 mL) and deionized water (500 mL), then eluted with acetonitrile (100 mL), then methanol (50 mL). Solvents were removed by rotary evaporation under reduced pressure, with absolute ethanol (50 mL) added to azeotrope traces of water, and the residue was dried overnight in vacuo to provide mu-oxo bis[iron (III)-4,5-diethyl-16,17-bis(2-[2-(2-methoxyethoxy)ethoxy]ethoxy)-9,24-bis(3-acetoxypropyl)-10,23-dimethyl-13,20,25,26,27-pentaazapentacyclo[20.2.1.1^{3,6}.1^{8,11}.0^{14,19}]heptacosa-1,3,5,7,9,11(27),12,14,16,18,20,22(25),23-tridecaene], i.e., Fe(Tex)₂O, as a brown/green powder (1.145 g, 53%). Positive ESI MS (methanol), M⁺: m/e 937 (calc. for [C₉₆H₁₃₂N₁₀O₂₁Fe₂]²⁺, 936.4). UV/vis (CH₃OH) [λₘₐₓ, nm (log ε)]: 224 (4.63), 268 (4.62), 342 (4.76), 408 (4.90), 450 (4.88), 732 (4.34). Magnetic moment (Evans): 6.01 B.M. (per monomer).

### Example (1d)

### Preparation of Other Metal Complex of Formula (I)

The methods described above are used to obtain metal complexes of Formula (I) wherein the metal is Ce (III), Eu (II), Ni (II), Sm (II) and Yb (II).

### Example 2

### Pharmaceutical Formulations of Formula (I)

Example (2a): Perfusion Fluid

| Ingredient | Parts by Weight | |
|---|---|---|
| Compound of Formula (I) | 20 | mg |
| Mannitol (U.S.P.) | 50.0 | g |
| Sodium Dihydrogen Phosphate | 4.59 | g |
| Sodium Monohydrogen Phosphate | 6.53 | g |

| | |
|---|---|
| Water For Injection (U.S.P.) | q.s. to 1000 mL |

Wherein the ingredients are dissolved in a portion of the Water For Injection, and once dissolved, the remaining volume is made up with water for injection.

Example (2b): Injectable Preparation

| Ingredient | Amount |
|---|---|
| Compound of Formula (I) | 2.0 mg/mL |
| (e.g., Compound 1) | |
| Mannitol (U.S.P.) | 50.0 mg/mL |
| Gluconic acid (U.S.P.) | q.s. (pH 5-6) |
| Water For Injection (U.S.P.) | q.s. to 1.0 mL |
| Nitrogen gas (NF) | q.s. to cover |

Other compounds of Formula (I), such as those prepared in accordance with Example 1, can be used as the active compound in the preparation of the formulations of this example.

### Example 3

### In Vivo Ischaemia Studies

A. Protection Against Cardiac Ischaemia

Myocardial protective activity of compounds of Formula (I) can be tested using an adaptation of the test described by Alps, et al., (Arzneim. Forsch Drug Res., 33, (1), 6, 868-876, 1983).

Eight male baboons are anaesthetized then randomly allocated to one of two groups. Group A (control group)—Four animals are subjected to 30 min. occlusion of the left anterior descending coronary artery (LAD) followed by a reperfusion period of 5.5 hours. Venous plasma samples taken pre-thoracotomy, pre-LAD ligation and every hour during the reperfusion period are analyzed for CPK₂ and LDH₁ iso-enzyme levels. Group B (treated group)-Treated as in group A, except that the animals receive a loading dose of test compound (5 μg/kg) intravenously 10 min. before LAD ligation followed by a continuous infusion of 0.05 μg/kg/min. for a 6-hour period starting at LAD ligation time.

Lower CPK₂ and LDH₁ iso-enzyme levels in Group B as compared to those measured in Group A, particularly at the 6-hour time point, are indicative of protection of the myocardial tissue against the deleterious effects of ischaemia.

B. Skeletal Muscle Protection

Skeletal muscle protective activity of Formula (I) compounds can be determined using experimental conditions described in Example 3A, except that plasma samples are assayed for CPK₃ and LDH₅ iso-enzymes. Lower CPK₃ and LDH₅ iso-enzyme levels in Group B as compared to those measured in Group A are indicative of protection of skeletal muscle against the deleterious effects of ischaemia, e.g., resulting from surgery-induced damage.

C. Protection Against Cerebral Ischaemia

Cerebral protective activity of the compounds of Formula (I) is determined under to the experimental conditions described in Example 3A, except that plasma samples are assayed for CPK₁ iso-enzyme. Lower CPK₁ iso-enzyme levels in Group B as compared to those measured in Group A are indicative of protection of the brain against the deleterious effects of ischaemia.

### Example 4

### Myocardial Protection During Cardioplegia

Myocardial protection activity of the compounds of Formula (I) against the sequelae of low flow perfusion can be tested using an adaptation of the method described by Ferrandon et al., Br. J. Pharmacol., 93, 247P, 1988.

Male Sprague-Dawley rats are anaesthetized with pentobarbitone sodium (50 mg/kg, i.p.). After injection of heparin (200 units i.v.) the thorax is opened, the heart removed with a length of aorta attached and then immersed in ice cold Krebs' solution (118 mM NaCl, 4.55 mM KCl, 1.2 mM KH₂SO₄, 1.2 mM MgSO₄, 11.0 mM glucose, 20.0 mM NaHCO₃, 1.35 mM CaCl₂, pH 7.4). The heart is gently palpated to expel the blood. Hearts are then perfused with the above solution warmed to 37° C. and gassed with 95% O₂ and 5% CO₂ retrogradely via the aorta (Langendorff model) using a peristaltic pump set to deliver 14 ml/min. A microelectrode is introduced into the ventricular muscle wall and a reference electrode placed in contact with the perfusion fluid 3 cm above the heart. The two electrodes are connected to a pH meter.

Hearts are perfused at 14 ml/min for a 15 min period to obtain a stable baseline ventricular pH. The aortic flow is then reduced to 1 ml/min for 15 min by decreasing the pump speed. The flow is then restored to the initial rate for 15 min. Values of coronary flow and ventricular pH are measured at 5-minute intervals. After restoration of the initial flow rate measurements are made at 30 seconds, 1 minute and 5 minutes. Samples of coronary effluent are collected and stored on ice. In a test group, infusions of test compound (1 μM) are started 10 min prior to reducing the flow rate and continued for the remainder of the experiment. At the end of the experiment the atria are removed and the hearts dried at 75° C. for 2 days.

Biochemical determination of lactate released into the coronary effluent is made using a spectrophotometric method. The quantity of lactate contained in the samples is obtained by reference to a standard curve. Lactate release from the heart mass is calculated using the following formula:

$$\frac{[ lactate ] ( μ M \text{/} mL ) \times coronary flow ( mL \text{/} min )}{dry weight of the heart ( g )}$$

Inhibition of fall in pH and lactate release levels in the test group as compared to those measured in the control group are indicative of myocardial protection against the sequelae of low flow perfusion.

### Example 5

### Protection For Organ Transplants

Organ transplant protection activity of compounds of Formula (I) can be determined using the procedure described below.

Twenty left nephrectomised pigs are autotransplanted with their kidneys after preservation for 24 hours in phosphate buffered sucrose (PBS 140) and immediate contralateral nephrectomy followed the autotransplantation. The quality of the preservation and post-transplant renal function is assessed by measurement of glomerular filtration rate (GFR) using insulin clearance on day 7.

Group A (n=10) placebo group—The animals receive placebo pre-treatment (bolus and infusion) commencing 5 min prior to left nephrectomy and lasting until the kidney is removed. The kidney is then flushed with PBS 140 containing placebo before storage in PBS 140. After 24 hours storage the kidney is auto-transplanted.

Group B (n=10) treated group—The animals receive a bolus dose of test compound intravenously (5 μg/kg) 5 min prior to nephrectomy followed by an infusion (0.05 μg/kg/h) until the kidney is removed. The kidney is then flushed with PBS 140 solution containing the test compound 0.5 μg/L (made up imediately before flush) prior to storage. After 24 hours storage the kidney is auto-transplanted.

Higher GFR at day 7, particularly with lower peak serum urea and peak serum creatinine levels in the test vs. control group are indicative of graft protection.

### Example 6

### Determination of Anti-Inflammatory Activity Utilizing Adjuvant-Induced Arthritis In The Rat

Anti-inflammatory activity of compounds of Formula (I) can be determined using a modified procedure initially described by Pearson, C. M., Proc. Soc. Exp. Biol. Med., 91: 95-101 (1956).

Female Simonsen albino rats weighing 160-180 g receive 0.1 ml of a suspension in paraffin oil of heat-killed M. Mycobacterium butyricum (10 mg/ml) by means of an intradermal injection into the proximal 1/4 of the tail on day 0. Beginning on day 1, the test material is administered orally in an aqueous vehicle (0.5 ml/dose) twice each day for 17 days. On day 18 the intensity of the swelling of the four foot pads and tail is determined utilizing a scoring system in which the swelling in the four paws was scored 04 for each paw and the tail swelling is scored 0-3, such that the total maximum score is 19.

### Example 7

### Determination of Autoimmune Activity Utilizing Experimental Allergic Encephalomyelitis

Autoimmune activity of Formula (I) compounds can be determined using a modification of a procedure initially described by Grieg, et al., J. Pharmacol. Exp. Ther. 173: 85 (1970).

On day 1, Experimental Allergic Encephalomyelitis is induced by giving an 0.1 ml sub-plantar injection into the dorsum of the right hind paw of an emulsion consisting of 15 mg (wet weight) of syngeneic spinal cord tissue, 0.06 ml of Fruend's Incomplete Adjuvant (Difco), 0.04 ml of sterile 0.9% saline, and 0.2 mg of heat killed and dried Mycobacterium butyricum (Difco). On days 12-17, clinical evaluations are obtained for each animal. The animals are considered positive if flaccid hind limb paralysis is present on one or more days.

### Example 8

### Determination of Immunosuppressive Activity Utilizing The Hemolytic Plaque Forming Cell Assay

Immunosuppressive activity of Formula (I) compounds can be determined using a modification of “The agar plaque technique for recognizing antibody producing cells,” a procedure initially described by Jerne, et al. [Cell-bound Antibodies, Amos and Kaprowski editors (Wistar Institute Press, Philadelphia, 1963) p. 109].

Groups of 5-6 adult C578B1/6 male mice are sensitized with 1×10⁸ sheep red blood cells (“SRBC”) and simultaneously treated with an oral dosage form of the test material in an aqueous vehicle. Animals in a control group receive the same volume of vehicle. Four days after SRBC inoculation, spleens are dispersed in loose Ten Broeck homogenizers. The number of nucleated cells (“WBC”) is determined and the spleen cell suspension is mixed with SRBC, guinea pig complement and agar solution at 0.5% concentration. Aliquots of the above mixture (0.1 ml) are dropped on four separate quadrants of a Petri dish and are covered with cover slips. After two hours incubation at 37° C., areas of hemolysis around plaque-forming cells (“PFC”) are counted with a dissecting microscope. Total WBC/spleen, PFC/spleen and PFC/10.sup.6 WBC (“PPM”) are calculated for each mouse spleen. Geometric means of each treatment group are then compared with the vehicle-treated control group.

### Example 9

### Determination of Immunosuppressive Activity Utilizing Responses of Human Peripheral Blood Lymphocytes to T- and B-cell Mitogens

Immunosuppressive activity of Formula (I) compounds can also be determined using a modification of a procedure initially described by Greaves, et al. [“Activation of human T and B lymphocytes by polyclonal mitogens,” Nature, 248, 698-701 (1974)].

Human mononuclear cells (“PBL”) are separated from heparinized whole blood by density gradient centrifugation in Ficoll-Paque (Pharmacia). After washing 2×10⁵ cells/well are cultured in microfiter plates with RPMI 1640 supplemented with 5% fetal calf serum, penicillin and streptomycin. To evaluate differential effects on T- and B-lymphocytes, different mitogens are used: PHA (Sigma) at 10 μg/ml, PWM (Sigma) at 20 μg/ml and Staphylococcus Protein A bound to Sepharose (SPA) (Sigma) 2 mg/ml or 14 μg/ml of Protein A. Test materials are tested at concentrations between 10⁴ and 10⁸ M, by addition to the culture at time 0. Cultures are set up in quadruplicate and incubated at 37° C. in a humidified atmosphere with 7% CO₂ for 72 hours. A pulse of 0.5 μCi/well of ³H-thymidine is added for the last 6 hours. Cells are collected on glass fiber filters with an automatic harvester and radioactivity is measured by standard scintillation procedures. The 50% inhibitory concentration (“IC₅₀”) for mitogenic stimulation is determined graphically.

### Example 10

### Efficacy Analysis of Motexafin Gadolinium (MGd) as a Neuroprotective Agent in the G93A Murine ALS Model Using a Loading Dose of 5 mg/kg and 2.5 mg/kg/day Lp. Thereafter

Transgenic mice which overexpress the G93A human Cu, Zn superoxide dismutase (SOD 1) mutant develop motor paralysis similar to amyotrophic lateral sclerosis (ALS) in humans. At 90-100 days of age, these mice develop hindlimb weakness which rapidly progresses to total body paralysis within 2-3 weeks. Such trangenic mice were used to evaluate the survival and neuroprotective action of MGd with G93A transgenic mice. In particular, the purpose of this study was to test the effect of MGd (administered at a loading dose of 5 mg/kg and a maintenance dose of 2.5 mg/kg/day i.p. thereafter).

Studies involved the use of G93A overexpressing mice purchased directly from JAX Labs. Due to the high variability in age of onset (of motor neuron disease or MND) and total survival, a mouse colony was bred using the JAX Labs protocols. Transgenic littermates were found to develop MND within a day or so of each other and progress toward total body paralysis at very similar rates such that their ultimate survival was also very similar. Therefore, littermate males were chosen to sire subsequent offspring and used to produce 10-12 litters of age-matched mice for drug studies. Typically 50-60 transgenic mice were obtained from 10-12 litters and, within these groups, the variability in age of onset and survival was quite low.

The procedure used involved monitoring the transgenic mice from several litters for onset of symptoms of motor neuron degeneration. At symptom onset (muscle weakness), mice were randomly assigned sequentially to either Control (no treatment) or Treatment (MGd-treated) groups. Mice were sacrificed when moribund (unable to assume upright posture), as required by animal welfare protocol. Motexafin gadolinium formulated at 2 mM (2.3 mg/ml) in 5% aqueous mannitol was used for this study and the study group involved 10-11 animals in each group, 21 animals total.

Group #1: Control group of 11 animals.

Group #2: Treatment group of 10 animals.

At the onset of symptoms MGd was administered via i.p injection at a loading dose of 5 mg/kg and a maintenance dose of 2.5 mg/kg/day i.p injection. was administered thereafter.

The reuslts are shown below in a Kaplan-Meier survival curve (plot A) and the survival interval of control and MGd-treated G93A mice is shown in plot B below.

[image]

The survival data used for plot B is given in the table below.

| | | | | | | | | | | | |
|---|---|---|---|---|---|---|---|---|---|---|---|
| Control | 12 | 16 | 16 | 13 | 14 | 12 | 13 | 12 | 14 | 12 | 14 |
| MGd | 53 | 26 | 23 | 21 | 20 | 46 | 55 | 36 | 32 | 54 | |

A significant survival effect with onset administration of MGd, using a loading dose of 5 mg/kg i.p., and 2.5 mg/kg/day i.p. thereafter was obtained, wherein MGd extended mean survival roughly 2.7-fold over untreated control mice (36.6 vs 13.4 days, p-value=4×10⁻⁵, non-paired T-Test).

### Example 11

### Efficacy Analysis of Motexafin Gadolinium (MGd) as a Neuroprotective Agent in the G93A Murine ALSModel Using a Dose of 1 mg/kg/day i.p.

Transgenic mice which overexpress the G93A human Cu, Zn superoxide dismutase (SOD1) mutant develop motor paralysis similar to amyotrophic lateral sclerosis (ALS) in humans. At 90-100 days of age, these mice develop hindlimb weakness which rapidly progresses to total body paralysis within 2-3 weeks. Such trangenic mice were used to evaluate the survival and neuroprotective action of MGd with G93A transgenic mice. In particular, the purpose of this study was to test the effect of MGd (administered at a dose of 1 mg/kg/day i.p.).

Studies involved the use of G93A overexpressing mice purchased directly from JAX Labs. Due to the high variability in age of onset (of motor neuron disease or MND) and total survival, a mouse colony was bred using the JAX Labs protocols. Transgenic littermates were found to develop MND within a day or so of each other and progress toward total body paralysis at very similar rates such that their ultimate survival was also very similar. Therefore, littermate males were chosen to sire subsequent offspring and used to produce 10-12 litters of age-matched mice for drug studies. Typically 50-60 transgenic mice were obtained from 10-12 litters and, within these groups, the variability in age of onset and survival was quite low.

The procedure used involved monitoring the transgenic mice from several litters for onset of symptoms of motor neuron degeneration. At symptom onset (muscle weakness), mice were randomly assigned sequentially to either Control (no treatment) or Treatment (MGd-treated) groups. Mice were sacrificed when moribund (unable to assume upright posture), as required by animal welfare protocol. Motexafin gadolinium formulated at 2 mM (2.3 mg/ml) in 5% aqueous mannitol was used for this study and the study group involved 9 animals in each group, 18 animals total.

Group #1: Control group of 9 animals.

Group #2: Treatment group of 9 animals.

At the onset of symptoms MGd was administered via i.p injection at a dose of 1 mg/kg/day.

The reuslts are shown below in a Kaplan-Meier survival curve (plot A) and the survival interval of control and MGd-treated G93A mice is shown in plot B below.

[image]

The survival data used for plot B is given in the table below.

| | | | | | | | | | |
|---|---|---|---|---|---|---|---|---|---|
| Control | 14 | 17 | 19 | 17 | 14 | 16 | 16 | 20 | 22 |
| MGd | 48 | 41 | 41 | 35 | 49 | 35 | 39 | 52 | 54 |

A significant survival effect with onset administration of MGd, using a dose of 1 mg/kg/day i.p. was obtained, wherein MGd extended mean survival roughly 2.5-fold over untreated control mice (43.8 vs 17.2 days, p-value<1×10⁻⁵, non-paired T-Test).

### Example 12

### Efficacy Analysis of Motexafin Gadolinium (MGd) as a Neuroprotective Agent in the G93A Murine ALS Model Using a Dose of 25 mg/kg/q.2d. and 2.5 mg/kg/q.4d. i.p.

Transgenic mice which overexpress the G93A human Cu, Zn superoxide dismutase (SOD 1) mutant develop motor paralysis similar to amyotrophic lateral sclerosis (ALS) in humans. At 90-100 days of age, these mice develop hindlimb weakness which rapidly progresses to total body paralysis within 2-3 weeks. Such trangenic mice were used to evaluate the survival and neuroprotective action of MGd with G93A transgenic mice. In particular, the purpose of this study was to test the effect of MGd (administered at a dose of 2.5 mg/kg/q.2d. and 2.5 mg/kg/q.4d. i.p.).

Studies involved the use of G93A overexpressing mice purchased directly from JAX Labs. Due to the high variability in age of onset (of motor neuron disease or MND) and total survival, a mouse colony was bred using the JAX Labs protocols. Transgenic littermates were found to develop MND within a day or so of each other and progress toward total body paralysis at very similar rates such that their ultimate survival was also very similar. Therefore, littermate males were chosen to sire subsequent offspring and used to produce 10-12 litters of age-matched mice for drug studies. Typically 50-60 transgenic mice were obtained from 10-12 litters and, within these groups, the variability in age of onset and survival was quite low.

The procedure used involved monitoring the transgenic mice from several litters for onset of symptoms of motor neuron degeneration. At symptom onset (muscle weakness), mice were randomly assigned sequentially to either Control (no treatment) or Treatment (MGd-treated) groups. Mice were sacrificed when moribund (unable to assume upright posture), as required by animal welfare protocol. Motexafin gadolinium formulated at 2 mM (2.3 mg/ml) in 5% aqueous mannitol was used for this study and the study group involved 5-6 animals in each group, 17 animals total.

Group #1: Control group of 5 animals.

Group #2: Treatment group of 6 animals. MGd administered at a dose of 2.5 mg/kg/q.2d. i.p., at the onset of symptoms.

Group #3: Treatment group of 6 animals. MGd administered at a dose of 2.5 mg/kg/q.4d. i.p., at the onset of symptoms.

The results are shown below in a Kaplan-Meier survival curve (plot A) and the survival interval of control and MGd-treated G93A mice is shown in plot B below.

[image]

The survival data used for plot B is given in the table below.

| Control | 23 | 20 | 28 | 19 | 26 | |
|---|---|---|---|---|---|---|
| MGd q2d | 20 | 37 | 35 | 30 | 28 | 28 |
| MGd q4d | 30 | 38 | 20 | 35 | 26 | 47 |

A trend towards increased mean survival with onset administration of MGd, using a dose of 2.5 mg/kg/q.2d. and 2.5 mg/kg/q.4d. i.p., wherein MGd extended survival roughly 1.3 to 1.4-fold over untreated control mice (30, 33 vs. 23 days, p-value=0.069, 0.068, non-paired T-Test).

### Example 13

### Efficacy Analysis of Motexafin Gadolinium (MGd) as a Neuroprotective Agent in the G93A Murine ALS Model Using a Dose of 1 mg/kg/day Lp. in Combination with the Administration of a Glutamate Antagonist

Transgenic mice which overexpress the G93A human Cu, Zn superoxide dismutase (SOD1) mutant develop motor paralysis similar to amyotrophic lateral sclerosis (ALS) in humans. At 90-100 days of age, these mice develop hindlimb weakness which rapidly progresses to total body paralysis within 2-3 weeks. Such trangenic mice are used to evaluate the survival and neuroprotective action of MGd with G93A transgenic mice in combination with the administration of a glutamate antagonist. In particular, the purpose of this study is to test the effect of MGd (administered at a dose of 1 mg/kg/day. in combination with the administration of a glutamate antagonist at 1 mg/kg/day).

Studies involve the use of G93A overexpressing mice purchased directly from JAX Labs. Due to the high variability in age of onset (of motor neuron disease or MND) and total survival, a mouse colony is bred using the JAX Labs protocols. Transgenic littermates are found to develop MND within a day or so of each other and progress toward total body paralysis at very similar rates such that their ultimate survival as also very similar. Therefore, littermate males are chosen to sire subsequent offspring and used to produce 10-12 litters of age-matched mice for drug studies. Typically 50-60 transgenic mice are obtained from 10-12 litters and, within these groups, the variability in age of onset and survival as quite low.

The procedure used involves monitoring the transgenic mice from several litters for onset of symptoms of motor neuron degeneration. At symptom onset (muscle weakness), mice are randomly assigned sequentially to either Control (no treatment) or Treatment (MGd/glutamate antagonist-treated) groups. Mice are sacrificed when moribund (unable to assume upright posture), as required by animal welfare protocol. Motexafin gadolinium formulated at 2 mM (2.3 mg/ml) in 5% aqueous mannitol is used for this study and the study group involved 9 animals in each group, 18 animals total.

Group #1: Control group of 9 animals.

Group #2: Treatment group of 9 animals.

The results will demonstrate an improved survival rate and neuroprotection in comparison with the control group.

What is claimed is:

1. A method of treating a neurologic disorder in a patient comprising administering to the patient an effective amount of a complex having the structure: 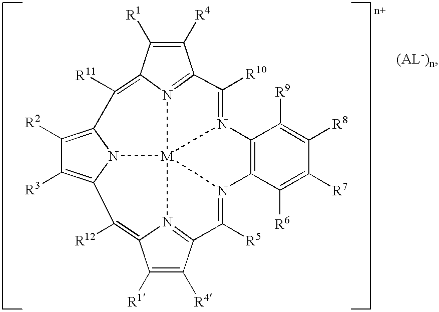
wherein:
M is a lanthanide metal ion,
AL is an apical ligand;
n is 1, 2, 3, 4, or 5;
R⁶ and R⁹ are independently chosen from the group: acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, sulfanyl, sulfinyl, sulfonyl, and the moiety —X-Y where: X is a covalent bond or a linker, and Y is a catalytic group, a neuroprotectiv agent or a site-directing group;
R¹, R^{1′}, R², R³, R⁴, R^{4′}, R⁷ and R⁸ are independently chosen from the group: acyl, acyloxy, alkyl, optionally substituted alkenyl, optionally substituted alkoxy, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, sulfanyl, sulfinyl, sulfonyl, and the moiety —X-Y where: X is a covalent bond or a linker, and Y is a catalytic group, a neuroprotective agent or a site-directing group; and
R⁵, R¹⁰, R¹¹ and R¹² are independently chosen from the group: acyl, optionally substituted alkoxy, optionally substituted alkyl, optionally substituted aryl, halo, and hydrogen;
with the proviso that for R⁶ and R⁹, halogen is other than iodide and substituted alkyl is other than iodoalkyl; and with the proviso that at least one of R¹, R^{1′}, R², R³, R⁴, R⁴′, R⁷ and R⁸ is —O-(optionally substituted alkylene-O)ₙ-alkyl, where n is 1, 2, 3, 4, 5, 6, 7, 8, 9, or 10.

2. The method of claim 1, wherein at least two of R¹, R^{1′}, R², R³, R⁴, R⁴′, R⁷ and R⁸ are —O-(optionally substituted alkylene-O)ₙ-alkyl, where n is 1, 2, 3, 4, 5, 6, 7, 8, 9, or 10.

3. The method of claim 1, wherein R⁵, R¹⁰, R¹¹ and R¹² are H.

4. The method of claim 1, wherein at least two of R¹, R^{1′}, R², R³, R⁴, R⁴′, R⁷ and R⁸ are unsubstituted alkyl.

5. The method of claim 1, wherein at least four of R¹, R^{1′}, R², R³, R⁴, R⁴′, R⁷ and R⁸ are unsubstituted alkyl.

6. The method of claim 1, wherein R⁷ and R⁸ are —O-(alkylene-O)ₙ-alkyl, where n is 1, 2, 3, 4, 5, 6, 7, 8, 9, or 10.

7. The method of claim 6, wherein n is an integer selected from 2, 3, 4, or 5.

8. The method of claim 7, wherein n is 3.

9. The method of claim 1, wherein R⁶ and R⁹ are hydrogen.

10. The method of claim 1, wherein R⁵, R¹⁰, R¹¹ and R¹² are hydrogen.

11. The method of claim 1, wherein AL is derived from any molecule containing a carboxylic acid or phosphate group.

12. The method of claim 11, wherein AL is acetate.

13. The method of claim 1, wherein M is selected from the group consisting of lanthanum, cerium, praseodymium, neodymium, promethium, samarium, europium, gadolinium, terbium, dysprosium, holmium, erbium, thulium, ytterbium and lutetium.

14. The method of claim 13, wherein M is Ce(III), Sm(II), Sm(III), Eu(II), Eu(III), Gd(III), Yb(II), Yb(III) and Lu(III).

15. The method of claim 1, wherein the complex decreases intracellular reactive oxygen species.

16. The method of claim 15, wherein said reactive oxygen species is OH, H₂O₂, O₂.—or ⁻OONO.

17. The method of claim 15, wherein the presence of said reactive oxygen species is associated with a disease.

18. The method of claim 1, wherein the administration of said complex results in the prevention, arresting or treatment of said disease.

19. The method of claim 1, wherein said disease is amyotrophic lateral sclerosis, Alzheimer's disease, Parkinsons disease, multiple sclerosis, and Huntington's disease.

20. The method of claim 1, wherein the complex has myocardial protective activity, skeletal muscle protective activity, or cerebral protective activity.

21. The method of claim 1, wherein the complex is administered in a solution.

22. The method of claim 21, wherein said complex is administered intravenously.

23. The method of claim 21, wherein said complex is administered in a solution containing about 2-8% of mannitol.

24. The method of claim 21, wherein the pH of the solution is between about 5 and 6.

25. The method of claim 1, wherein said complex is co-administered with an antiemetic.

26. The method of claim 1, wherein the complex is administered in multiple doses.

27. The method of claim 1, wherein the patient is further administered with an agent selected from a thrombolytic agent, an anti-anginal agent a reducing agent, another neurological therapeutic agent, or a zinc compound.

28. The method of claim 1, wherein the complex has the structure:

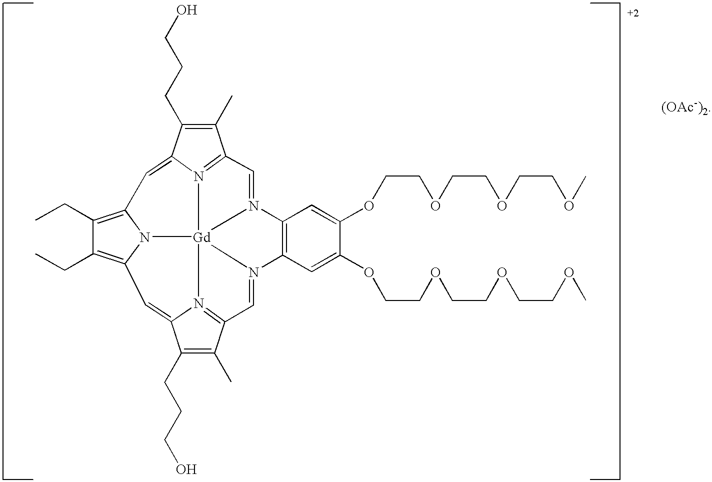

29. The method of claim 1, wherein the complex has the structure: [chemical structure]
